(19) United States
(12) Patent Application Publication
(10) Pub. No.: US 2012/0040976 A1
(43) Pub. Date: Feb. 16, 2012

(54) SMALL MOLECULE IMMUNOMODULATORS FOR ALZHEIMER'S DISEASE

(75) Inventors: John R. Cashman, San Diego, CA (US); Kenneth J. Abel, San Diego, CA (US)

(73) Assignee: HUMAN BIOMOLECULAR RESEARCH INSTITUTE, San Diego, CA (US)

(21) Appl. No.: 13/139,274

(22) PCT Filed: Dec. 11, 2009
(86) PCT No.: PCT/US2009/067791

§ 371 Date: Aug. 24, 2011

Related U.S. Application Data
(60) Provisional application No. 61/201,546, filed on Dec. 11, 2008.

(51) Int. Cl. A61K 31/5377; A61K 31/136; A61K 31/122; A61K 31/225; A61K 31/4025; G01N 33/566; A61K 31/4545; A61P 25/28; A61P 25/00; C12Q 1/68; C12Q 1/48; A61K 31/12; A61K 31/4155

(52) U.S. Cl. 514/231.8; 514/679; 514/646; 514/683; 514/684; 514/548; 514/422; 514/406; 514/316; 435/6.13; 435/6.11; 435/6.12; 435/15; 435/7.21

(57) ABSTRACT

Disclosed are methods for identifying individuals suffering from a CNS disorder (including Alzheimer's Disease, ALS, behavioral disorders, and the like) that could be treated with a CNS drug with greater therapeutic efficacy and lower side effects and the compounds useful for such treatment. Also disclosed are methods for predicting the efficacy of a drug candidate for the treatment of a CNS disorder. The technology is also applicable to drug discovery for evaluation in animal models of neurodegenerative diseases.

### CROSS-REFERENCE TO RELATED APPLICATIONS

This application claims benefit to U.S. Application Ser. No. 61/201,546 filed Dec. 11, 2009, which is incorporated herein by reference in its entirety.

### FIELD OF INVENTION

The invention relates to small molecule immunostimulants useful to treat individuals with clinically characterized Alzheimer's Disease (AD) or Amyotrophic lateral sclerosis (ALS). The small molecule immunostimulants consist of compounds that interact with genes and proteins that are associated with significantly elevated clinical efficacy of AD and ALS medications including curcumin and curcumin analogs, and related immune modulators. Also provided are compounds capable of up-regulation of N-acetylglucosaminyltransferase III (MGAT3), vitamin D3 receptor (VDR) and Toll-like receptors (TLRs), increasing phagocytosis of amyloid-β (1-42) (Aβ), and suppression of neuro-cytotoxic inflammatory agents. Further provided is a method for detecting regulation of MGAT3, VDRs and TLRs or MGAT3, VDRs or TLR polymorphic variants, and quantifying the potential for AD and ALS or other neurodegenerative diseases in biological samples.

### BACKGROUND OF THE INVENTION

Enhancement of the Innate Immune System

Treatment of Alzheimer's disease remains an elusive goal, because current therapies are not effective, animal models are poor and the underlying pathology is not understood. Diagnosis of the disease early in progression is very difficult. Abeta (Aβ) accumulation in AD brain is related to abnormal communication between Aβ reactive T cells and microglia leading to differentiation of microglia into either phagocytes or antigen presenting cells and inhibition of complement activation (Science 302 (2003) 834-838). It was shown that macrophages and microglia of middle-aged and older normal subjects perform Aβ clearance, but this function is defective in AD patients (Journal of Alzheimer's Disease 7 (2005) 221-232) although no defect for AD patients has been detected in bacterial phagocytosis.

Chemical substances such as curcuminoids, VDR agonists, and the hormone insulin-like growth factor (IGF-I) can bolster the innate immune system. In some cases, there is epidemiologic and aging-related rationale for use of these agents to treat AD. Aβ uptake and degradation by AD macrophages is significantly lower in comparison to control macrophages. In some cases, AD macrophages participate in surface binding but not intracellular phagocytosis of Aβ. After treatment of AD macrophages with curcuminoids or curcuminoid analogs, Aβ uptake by macrophages of AD patients increases and increase of phagocytosis occurs. Therefore, enhancement of innate immunity may provide a natural non-toxic approach to AD therapy.

Activation of microglia is considered to be responsible for both brain inflammation and Aβ phagocytosis through various receptors. Immunohistochemical studies of AD brain showed that inducible nitric-oxide synthase (NOS)-positive and cyclooxygenase-2 (COX-2)-positive blood-borne monocyte/macrophages penetrate across brain microvessels and infiltrate perivascular and parenchymal sites, but only partially clear neuritic plaques. The data show that in the human AD brain, peripheral monocyte/macrophages are involved in Aβ clearance. It was also shown that peripheral blood macrophages and T-cells are able to invade the brain of aged amyloid precursor protein transgenic (APP23) mice and afford clearance of Aβ deposits.

Currently, there is no clinically successful strategy to remove and degrade Aβ deposits from the brain. In sporadic cases of AD, amyloidosis of the brain may be related to defective clearance of Aβ. This has led to development of an Aβ vaccine, but its use in a clinical trial was abrogated due to adverse encephalitic complications. But such an Aβ vaccine does not address the fundamental problem of degrading and removing the plaque.

Studying the benefits of enhancing the immune response to Aβ using peripheral blood leukocytes of AD patients and control subjects has significant advantages. In cultured macrophages of AD patients in vitro, curcuminoids improve the defect in macrophage phagocytosis of Aβ of about 75% of the patients studied. This mechanism of action of curcuminoids is novel and may be independent of the known anti-inflammatory and pro-apoptotic effects of curcuminoids. It is also shown that up-regulation of VDR occurs in parallel to the improvement of Aβ phagocytosis in macrophages.

The effects of immunomodulating therapies could be evaluated in vitro and individualized according to each subject's innate and adaptive immune responses. This requires information about genetic and biochemical markers of immune response that is described herein. As described below, curcuminoids up-regulate the MGAT3, VDR and TLR genes and this may be an important part of stimulating neuroprotective mechanisms with potential for treating both AD and ALS.

MGAT3, VDR and TLRs in Neurodegeneration

Nearly all proteins of blood serum and on cell surfaces of higher organisms are glycosylated. The N-glycans of mammalian glycoproteins vary widely in structure, but contribute to important biological processes. N-Acetylglucosaminyltransferase III (MGAT3), the product of the MGAT3 gene, transfers the bisecting GlcNAc to the core mannose of complex N-glycans. Defective MGAT3 could markedly change cell-mediated immunity and the function of other N-glycosylated biomolecules. Individuals with defective or abnormal amount of MGAT3 may have other neurobiological problems. Loss of MGAT3 or decreased expression over time may also have deleterious consequences and MGAT3 loss may compromise the normal cell processes including cytoprotection in AD and ALS.

The steroid hormone 1α,25(OH)-2-vitamin D3 (1,25D) and its metabolites collectively form nearly all of the small, lipophilic, molecules that regulate the complex vitamin D endocrine system (J Steroid Biochem Mol Biol 103 (2007) 243). Vitamin D sterols modulate these processes via regulating multiple tissue specific cellular signaling cascades via binding to the nuclear vitamin D receptor (VDR) and activating genomic and nongenomic cellular responses (Endocr Rev 16 (1995) 200; Mol Endocrinol 17 (2003) 777). Vitamin 1,25D appears to strongly stimulate phagocytosis and degradation of Aβ by AD patients' macrophages, with associated protection against apoptosis. Curcuminoids may show additive effects with vitamin 1,25D in stimulating Aβ phagocytosis and upregulation of certain genes (MGAT3, TLR's) in AD patients' macrophages. Cooperativity between curcuminoids and vitamin 1,25D may suggest a novel therapeutic approach to restoring defective innate immune function in AD patients toward clearance of brain Aβ plaque.

Toll-like receptors (TLRs) are a family of pattern-recognition receptors in the innate immune system. TLRs comprise a group of 10 genes and their gene products (i.e., TLR1-10). TLRs are cell-surface signaling receptors involved in host response. TLR agonists are being developed for the treatment of diseases that involve inappropriate adaptive immune diseases such as sepsis, autoimmune disease, cancer, allergies and viral and bacterial infections (Nat Med. 13, 552, 2007). TLR antagonists are being developed to combat inflammation and autoimmunity diseases. Most of the literature in this area has examined the role of inflammatory mediators in the activation of endogenous or exogenous microglia. For example, activation of microglia with a TLR ligand markedly boosts ingestion of Aβ in vitro (Tahara et al., Brain 129, 3006, 2006). Activation of TLR2 markedly enhanced mouse formyl peptide receptor-like 2 (mFPR2)-mediated uptake of Aβ by microglia (Chen et al., J. Biol. Chem. 281, 3651, 2006). Other studies have suggested that the TLR4 Asp299Gly variant may be protective against the development of late-onset AD.

Curcuminoids enhance uptake of Aβ by macrophages of AD patients. Normal subjects' macrophages perform adequately without any treatment. Treatment with curcuminoids enhances not only the intensity of uptake but also intracellular phagocytosis and transport to lysosomal compartments, reduces oxidative damage, interleukin-1 beta reactivity, and microgliosis in an APPs transgenic mouse model. Curcuminoids are also known to have anti-inflammatory properties and anti- and pro-apoptotic properties, which may modulate excessive inflammatory responses by macrophages. Anti-inflammatory properties may be of benefit for treating both AD and ALS. Other beneficial properties of curcuminoids, such as inhibiting aggregation of Aβ and possibly other proteins, are relevant to AD and ALS patients. However, despite their utility for treating these diseases, natural curcuminoids have very low bioavailability, are chemically and metabolically unstable, and possess very poor pharmaceutical properties.

Immunological studies shed new light on the immunopathogenesis of neurodegenerative diseases demonstrating the role of the innate immune system targeting misfolded proteins, such as amyloid-beta in AD and aggregated SOD-1 in ALS. Cyclooxygenase-2 (COX-2) and inducible NO synthase (iNOS)-positive macrophages infiltrate the ALS spinal cord encroaching into dying neurons. Recently, strong expression of COX-2 and other inflammatory cytokines has been observed in ALS monocytes and macrophages after stimulation with aggregated or mutant SOD-1. Anti-inflammatory drugs based on small molecule analogs of natural compounds (i.e. curcuminoids) likely suppress these responses if administered as a continuous therapy with beneficial effects to ALS patients.

ALS is a fatal neurodegenerative disease characterized by death of upper and lower motor neurons. 5-10% of ALS cases are familial associated with mutations in the Cu/Zn superoxide dismutase-1 (SOD-1) gene. A majority of ALS cases are considered sporadic, possessing wild type (WT) SOD-1. Their pathogenesis is suspected to be related to misfolded SOD-1 in neuronal inclusions with toxicity either directly to neurons, or through an inflammatory response by neighboring microglia, macrophages, or astrocytes. Toxic effects of misfolded proteins overwhelming the cellular disposal system are considered a key to the pathogenesis of neurodegenerative diseases. A common pathway of WT and mutant SOD-1 in ALS pathogenesis is likely related to aggregation, because SOD-1 mutations have diverse effects on the structure, activity and native stability of SOD-1 but all decrease the net charge of the protein promoting SOD-1 aggregation.

The enhancement of innate immune functions, phagocytosis and resistance to apoptosis, and suppression of inflammatory responses by curcuminoids suggests that immune modulation of the innate immune system with small molecules might be a safe alternative to vaccination. Therefore, the biochemical and functional defects of AD and ALS macrophages and their modulation by natural substances or their analogs provide an entirely new direction to the treatment of these diseases and create new diagnostic and therapeutic opportunities. Results with peripheral monocytes and macrophages suggest that treatment with small molecule curcumins or related agent immunostimulants in patients would be helpful to improve immunomodulatory properties and the pathogenesis of AD and ALS.

The human MGAT3, VDR and Toll-like receptor (TLR) genes may be useful in testing other immune modulators or other drug candidates for CNS drug activity or neurodegenerative diseases including treatment and diagnosis of AD, ALS and the like. The invention solves the problem of defects in phagocytosis of amyloid-β (1-42) (Aβ and related forms) and in clearance of Aβ plaques by AD patients, and of chronic inflammatory responses of the innate immune cells in ALS patients, by identifying highly potent curcuminoids and synthesizing more biologically active derivatives.

### SUMMARY OF THE INVENTION

In one aspect, provided are MGAT3, VDR and TLRs genes and corresponding proteins, fusion proteins and/or variant forms of these proteins as drug targets for modulation in vitro and/or in vivo as an indicator of CNS damage for a number of brain diseases or indicator of therapy. MGAT3, VDR or TLR modulation represents a promising approach to protect individuals suffering from AD, ALS or other neurodegenerative diseases.

In another aspect, evaluation of MGAT3, VDR and/or TLRs in isolated macrophages or modulation of MGAT3, VDR or TLRs in vivo or ex vivo offers a clinically relevant diagnostic and therapeutic tool and provides an immediate approach to neurodegenerative disease diagnosis and treatment.

In yet another aspect, provided are therapeutic agents (curcumins and/or analogs thereof) that can be used to up-regulate MGAT3, VDR and/or TLRs that facilitates Aβ plaque degradation and removal, and suppression of cytotoxic inflammatory agents. The compounds have the following formulas (I-III):

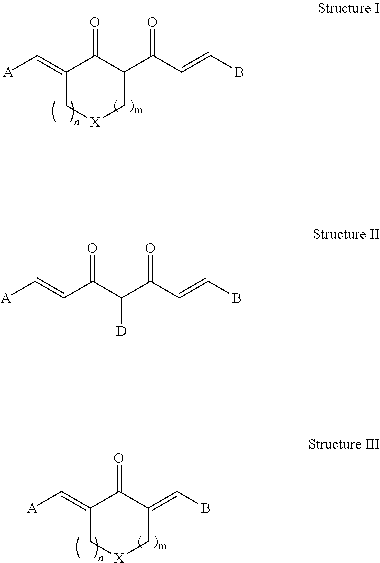

wherein A, B, D, X, n and m are as described below.

In another aspect, provided is a method for treating AD or ALS or other diseases by administering to a patient in need of such treatment a curcumin of the formula (I-III).

In another aspect, provided is a method for treating AD or ALS or other diseases by administering to a patient in need of such treatment a synthetic curcumin analog of the formula (I-III).

In another aspect, provided is a method for ex vivo modulation of MGAT3, VDR and/or TLRs comprising the steps of obtaining human blood cells, treating them with therapeutic agents and re-introducing the stimulated cells to stimulate Aβ plaque degradation and removal.

In another aspect, provided herein is a method to assess the profile of physiological, metabolic, genetic and biochemical signatures in human cells that can be derived and are predictive of the biological or physiological potential of a chemical or drug to promote human Aβ clearance. The instant invention solves the problem of predicting the potential of a chemical or drug as an anti-AD or ALS agent by identifying the effect on Aβ clearance at an early stage in an in vitro setting.

In another aspect, provided herein are novel agents capable of enhancing Aβ clearance.

In yet another aspect, provided are methods for in vitro screening of compounds for Aβ clearance potential or other biological activities by identifying biological parameters undergoing active change. These methods include incubating a chemical with a cell; determining the pathological, morphological and biochemical change and detection of the amount and type of cellular change produced.

In another aspect, provided are methods for in vitro screening of compounds for facilitating Aβ clearance potential or other biological activities of relevance to the in vivo condition.

In another aspect, provided is a method of predicting the potential of a chemical or drug as an anti-AD or anti-ALS agent by identifying its effect on Aβ clearance at an early stage in an in vitro setting. In another embodiment, provided is a method of identifying individuals that harbor defective or low levels of MGAT3, VDR or TLRs as biomarkers of use in predicting those individuals with AD, ALS or other neurodegenerative diseases.

In another aspect, provided is a method for predicting efficacy of an AD drug in an individual, where said drug is a MGAT3, VDR or TLR modulator and said individual is suffering from or at risk of developing a CNS disorder amenable to treatment with the drug, comprising the following steps: (1) isolating a biological sample from an individual, the biological sample comprising at least one of (i) nucleic acids and (ii) MGAT3 or VDR proteins (or general N-glycosylated proteins) or TLR; and (2) analyzing the biological sample to determine in the individual the presence or absence of MGAT3, VDR or TLR alleles or the MGAT, VDR or TLR gene, where the relative amount of expression of the MGAT3, VDR or TLR gene(s) is indicative of a positive clinical outcome for treatment of the disorder with the drug. In certain embodiments, the CNS disorder is a neurodegenerative disorder (e.g., AD or ALS). The methods are particularly suitable for use where, for example, drug has a relationship to anti-AD (e.g., the agent is a curcuminoid or analog). In one embodiment, the biological sample comprises nucleic acids. In another embodiment, the analyzing step comprises analyzing the nucleic acids from the biological sample to determine the nucleotides present in the MGAT3, VDR or TLR gene coding region. In yet another embodiment, the method can further include determining the MGAT3, VDR or TLR genotype at other nucleotide positions of the MGAT3, VDR or TLR gene coding region, non-coding region or promoter region. In another embodiment, the analyzing step comprises hybridization between said nucleic acid sample and a nucleic acid selected from the group consisting of (a) a nucleic acid comprising at least 10 to 100 contiguous nucleotides of the nucleotide sequences set forth in SEQ ID Nos: 1-12, where the nucleic acid includes the nucleotide at key MGAT3 or VDR or TLR alleles and/or a base adjacent thereto; and (b) a nucleic acid that is fully complementary to the nucleic acid of (a). In certain embodiments, the nucleic acid is conjugated with a detectable marker or agent to assist in isolation.

In another aspect, provided is a method for predicting the efficacy of a candidate agent for the treatment of a CNS disorder, where the candidate agent is a derivatized or modified form of a predetermined therapeutic agent for the treatment of the disorder, comprising the following steps: (1) contacting a first AD sample of an MGAT3, VDR or TLR protein with the candidate agent; (2) contacting a second normal sample of an MGAT3 or TLR protein with the predetermined therapeutic agent; where the contacting of each of the first and second samples is under conditions suitable for affording MGAT3 enzyme or VDR or TLR functional activity; (3) determining for each of the first and second samples the level of MGAT3 enzyme or VDR or TLR activity or DNA; and (4) comparing the level of MGAT3 enzyme or VDR or TLR activity or DNA in the first sample with the level of MGAT3 enzyme or VDR or TLR activity or DNA in the second sample, whereby a greater level of MGAT3 enzyme or VDR or TLR activity or DNA in the first sample relative to the second sample is indicative of efficacy of the candidate agent. In certain embodiments, a control used is the cDNA-expressed form of MGAT3 or VDR or TLR. In certain embodiments, the CNS disorder is a neurodegenerative disorder. In certain embodiments, the predetermined therapeutic agent is a curcuminoid or derivative or an immune-modulating agent. In certain embodiments, drug candidates are agents that have been derivatized to incorporate an MGAT3 or VDR substrate moiety (e.g., a curcuminoid-like center).

In some embodiments, the method for determining the level of MGAT3 enzyme or TLR activity comprises detecting the level of an N-glycosylated metabolite of the cell in a sample.

In another embodiment, provided is a method for ex vivo immunotherapy for patients with Alzheimer's Disease or ALS, involving PBMC's, and up-regulation of MGAT3, TLR's, or VDR by bisdemethoxycurcumin or other curcuminoids.

### BRIEF DESCRIPTION OF THE DRAWINGS

FIG. 1. Expression of MGAT3 in U-937 human monocytic cells in response to analog 33 alone, or followed by Aβ treatment. Real-time qPCR data were determined from duplicate reactions, and were normalized to expression data for the control gene HPRT. White bars, curcumin-only treated cells; black bars, curcumin plus Aβ treatment (+Aβ); grey bar, untreated cells.

FIG. 2. Expression of TLR2 in response to compound 10 alone, and in combination with Aβ.

FIG. 3. Expression of TLR3 in response to compound 10 alone, and in combination with Aβ.

FIG. 4. Expression of TLR4 in response to compound 10 alone, and in combination with Aβ.

FIG. 5. Expression of the VDR gene in response to analog 10 alone, or in combination with Aβ.

FIG. 6. Expression of selected genes in peripheral monocytes from young and aged AD and control mice. Each bar represents fold expression in AD relative to similarly aged control monocytes.

FIG. 7. Expression of ITR in selected tissues from young and aged AD and control mice. Each bar represents fold expression change in AD relative to similarly aged control tissues.

FIG. 8. Summary of two independent experiments evaluating a neuroprotective effect of curcumin (C) and bisdemethoxycurcumin (BDC) in a rat brain slice AD model. Slices were transfected with EYFP, hAPP, or EYFP+hAPP, and subsequently incubated with vehicle (DMSO) or compounds at various concentrations for 72 hrs.

FIG. 9. Normalized expression of MGAT3 and TLR4 genes in untransfected neuroblastoma cells (SHSY5Y), and in derived lines overexpressing wt or mutant SOD1. For comparisons, relative expression in untransfected SHSY5Y is set at ‘1’, and fold-expression differences in SOD⁺ cells are expressed relative to the control (untransfected) cells.

FIG. 10. Normalized expression of MGAT3 and TLR4 in fibroblast cell lines derived from control individuals, and from eight patients with either familial or sporadic ALS. Averaged data from the control, fALS, and sALS groups were compared. For comparisons, relative expression in control fibroblasts is set at ‘1’. Fold-expression differences in the ALS fibroblasts are expressed relative to control data.

FIG. 11. Normalized MGAT3 expression in neuroblastoma cell lines with either normal (NB) or overexpressed wt SOD1, exposed to H2O2 for 1-3 hours prior to gene expression studies. Fold-expression differences in the ALS fibroblasts are expressed relative to control data.

FIG. 12. Protective effect of BDC against oxidative stress. Cells were treated 2 hours in 0.1 mM H₂O₂ in varying concentrations of BDC, then stained with Trypan-blue to assess cell viability.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and material similar to those described herein can be used in the practice or testing of the present invention, only exemplary methods and materials are described.

For purposes of the present invention, the following terms are defined below where R refers to the R in Schemes 1 or 2.

The terms “a,” “an,” and “the” include plural referents unless the context clearly dictates otherwise.

The term “hydrido” refers to a single hydrogen.

The term “alkyl” refers to saturated aliphatic groups including straight chain, branched chain, and cyclic groups, all of which may be optionally substituted. Suitable alkyl groups include methyl, ethyl and the like, and may be optionally substituted.

The term “alkenyl” refers to unsaturated groups which contain at least one carbon-carbon double bond and includes straight chain, branched chain, and cyclic groups, all of which may be optionally substituted.

The term “alkynyl” refers to unsaturated groups which contain at least one carbon-carbon triple bond and includes straight chain, branched chain, and cyclic groups, all of which may be optionally substituted. Suitable alkynyl groups include ethynyl, propynyl, butynyl and the like which may be optionally substituted.

The term “aralkyl” refers to an alkyl group substituted with an aryl group. Suitable aralkyl groups include benzyl, and the like, and may be optionally substituted.

The term “alkoxy” refers to the ether —OR where R is alkyl, alkenyl, alkynyl, aryl, or aralkyl.

The term “aryloxy” refers to the ether —OR where R is aryl or heteroaryl.

The term “alkenyloxy” refers to ether —OR where R is alkenyl.

The term “alkylthio” refers to —SR where R is alkyl, alkenyl, alkynyl, aryl, aralkyl.

The term “alkylthioalkyl” refers to an alkylthio group attached to an alkyl thioether of about one to twenty carbon atoms through a divalent sulfur atom.

The term “alkylsulfinyl” refers to —S(O)R where R is alkyl, alkenyl, alkynyl, aryl, aralkyl.

The term “sulfonyl” refers to a —SO₂—R group where R is alkyl, alkenyl, alkynyl, aryl, or aralkyl.

The term “aminosulfonyl”, “sulfamyl”, “sulfonamidyl” refer to —SO₂NRR′ where R and R′ are independently selected from alkyl, alkenyl, alkynyl, aryl, and aralkyl.

The term “hydroxyalkyl” refers to linear or branched alkyl groups having one to about twenty carbon atoms any one of which may be substituted with a hydroxyl group.

The term “cyanoalkyl” refers to linear or branched alkyl groups having one to about twenty carbon atoms any one of which could be substituted with one or more cyano groups.

The term “alkoxyalkyl” refers to alkyl groups having one or more alkoxy groups attached to the alkyl group. The alkoxy group may be further substituted with one or more halo atoms. Preferred haloalkoxy groups may contain one to twenty carbons.

The term “oximinoalkoxy” refers to alkoxy groups having one to about twenty carbon atoms, any one of which may be substituted with an oximino group.

The term “aryl” refers to aromatic groups which have at least one ring having conjugated “pi” electron system and includes carbocyclic aryl, biaryl, both of which may be optionally substituted.

The term “carbocyclic aryl” refers to groups wherein the ring atoms on the aromatic ring are carbon atoms. Carbocyclic groups include phenyl and naphthyl groups which may be optionally substituted with 1 to 5 substituents such as alkyl, alkoxy, amino, amido, cyano, carboxylate ester, hydroxyl, halogen, acyl, nitro.

The term “aroyl” refers to —C(O)R where R is aryl group.

The term “alkoxycarbonyl” refers to —C(O)OR wherein R is alkyl, akenyl, alkynyl, aryl, or aralkyl.

The term “acyl” refers to the alkanoyl group —C(O)R where R is, alkenyl, alkynyl, aryl, or aralkyl.

The term “acyloxy” refers to the alkanoyl group —OC(O)R where R is, alkenyl, alkynyl, aryl, or aralkyl.

The term “aminoalkyl” refers to alkyl which is substituted with amino groups.

The term “arylamino” refers to amino groups substituted with one or more aryl groups.

The term “aminocarbonyl” refers to —C(O)NRR₁ wherein R and R₁ are independently selected from hydrogen, alkyl, akenyl, alkynyl, aryl, and aralkyl.

The azidoalkyl refers to alkyl R which is substituted with azido —N₃.

The term “amino” refers to —NRR₁ where R and R₁ are independently hydrogen, lower alkyl or are joined together to give a 5 or 6-membered ring such as pyrrolidine or piperidine rings which are optionally substituted.

The term “alkylamino” includes amino groups substituted with one or more alkyl groups.

The term “dialkylamino” refers to —NRR₁R and R₁ are independently lower alkyl groups or together form the rest of ring such as morpholino. Suitable dialkylamino groups include dimethylamino, diethylamino and morpholino.

The term “morpholinoalkyl” refers to alkyl R substituted with morpholine group.

The term “isocyanoalkyl” refers to alkyl R that is substituted with isocyano group —NCO.

The term “isothiocyanoalkyl” refers to alkyl R that is substituted with isothiocyano group —NCS.

The term “isocyanoalkenyl” refers to alkenyl R that is substituted with isocyano group —NCO.

The term “isothiocyanoalkenyl” refers to alkenyl R that is substituted with isothiocyano group —NCS.

The term “isocyanoalkynyl” refers to alkynyl R that is substituted with isocyano group —NCO.

The term “isothiocyanoalkynyl” refers to alkynyl R that is substituted with isothiocyano group —NCS.

The term “alkanoylamino” refers to —NRC(O)OR₁ where R and R₁ are independently hydrogen, lower alkyl, akenyl, alkynyl, aryl, or aralkyl.

The term “formyl” refers to —CHO attached to a nitrogen atom.

The term “optionally substituted” or “substituted” refers to groups substituted by one to five substituents, independently selected from lower alkyl (acyclic or cyclic), aryl (carboaryl or heteroaryl) alkenyl, alkynyl, alkoxy, halo, haloalkyl (including trihaloalkyl, such as trifluoromethyl), amino, mercapto, alkylthio, alkylsulfinyl, alkylsulfonyl, nitro, alkanoyl, alkanoyloxy, alkanoyloxyalkanoyl, alkoxycarboxy, (—COOR, where R is lower alkyl), aminocarbonyl (—CONRR₁, where R and R₁ are indepentyl lower alkyl), formyl, carboxyl, hydroxyl, cyano, azido, keto, and cyclic ketals thereof, alkanoylamido, heteroaryloxy, and heterocarbocyclicoxy.

The term “lower” refers herein in connection with organic functionality or compounds defined such as one up to and including ten, preferably up to and including six, and more preferably one to four carbon atoms. Such groups may be straight chain, branched chain, or cyclic.

The term “heterocyclic” refers to carbon containing groups having three, four, five, six, or seven membered rings and one, two, three, or four O, N, P, or S heteroatoms, e.g., thiazolidine, tetrahydrofuran, 1,4-dioxane, 1,3,5-trioxane, pyrrolidine, pyridyl, piperidine, quinuclidine, dithiane, tetrahydropyran, and morpholine or fused analogs containing any of the above.

The term “heteroaryl” refers to carbon containing 5-14 membered cyclic unsaturated groups containing one, two, three, or four O, N, P, or S atoms and having 6, 10 or 14 r electrons delocalized in one or more than one rings, e.g., pyridine, oxazole, indole, purine, pyrimidine, imidazole, benzimidazole, indazole, 2H-1,2-4-triazole, 1,2,3-triazole, 2H-1,2,3,4-tetrazole, 1H-1,2,3,4-triazolebenztriazole, 1,2,3-triazolo[4,5-b]pyridine, thiazole, isoxazole, pyrazole, quinoline, cytosine, thymine, uracil, adenine, guanine, pyrazine, picoline, picolinic acid, furoic acid, furfural, furyl alcohol, carbazole, isoquinoline, pyrrole, thiophene, furan, phenoxazine, and phenothiazine, each of which may be optionally substituted.

The term “amino acid” refers to natural and non-natural, L and D amino acids, as well a poly-amino acids.

The term “pharmaceutically acceptable esters, amides, or salts” refers to esters, amides, or salts of compounds of Scheme 1 derived from the combination of a compound of this invention and an organic or inorganic acid. Of particular interest are sulfate salts, which have shown improved solubility, particularly for those compounds having amine functionalities (e.g., compounds 3, 7, 9, 10, 13, 20, 31, 39, 41, 42, and 46-49).

The term “curcumin-related agent” refers to curcumin-related compounds, curcumin metabolites, curcumin analogues, and curcumin derivatives, including prodrugs as further described herein.

The term “inhibit” means to reduce by a measurable amount, or to prevent entirely.

“Treating,” “treatment,” or “therapy” of a disease or disorder means slowing, stopping, or reversing progression of the disease or disorder, as evidenced by a reduction or elimination of either clinical or diagnostic symptoms, using the compositions and methods of the present invention as described herein.

“Preventing,” “prophylaxis,” or “prevention” of a disease or disorder means prevention of the occurrence or onset of a disease or disorder or some or all of its symptoms.

The term “subject” as used herein means any mammalian patient to which the compositions of the present invention may be administered according to the methods described herein. Subjects specifically intended for treatment or prophylaxis using the methods of the present invention include humans.

The term “therapeutically effective regime” means that a pharmaceutical composition or combination thereof is administered in sufficient amount and frequency and by an appropriate route to at least detectably prevent, delay, inhibit, or reverse development of at least one symptom or biochemical marker of a neurodegenerative-related disorder. In certain embodiments, the “therapeutically effective regime” predisposes a subject to improve cognition, memory and other aspects of AD or ALS.

The term “therapeutically effective amount” refers to an amount of an anti-AD- or ALS-related agent, or a combination of a anti-AD- or ALS-related agent with other agent(s), to achieve a desired result, e.g., preventing, delaying, inhibiting, or reversing a symptom or biochemical marker of a neurodegenerative disorder or AD or ALS when administered in an appropriate regime.

“Amenable to treatment” with the drug means that the disorder is either predicted or determined to be a disorder that can be treated by administration of the drug (for example, through clinical testing such as by, e.g., clinical trials conducted to obtain governmental approval of a drug).

The term “positive clinical outcome” refers to any improvement, or decrease in frequency of, clinical symptoms associated with the disorder, as determined using known diagnostic methods. Generally, indication of a positive clinical outcome using the above method is indicative of greater efficacy of the drug in the individual relative to an individual in which the MGAT3, VDR or TLRs are absent.

MGAT3, VDR or TLR inducers or up-regulation moieties as used herein refer to any chemical moiety that is known or predicted to up-regulate, modulate or induce by interaction with the MGAT3, VDR or TLRs protein or gene during interaction of MGAT3, VDR or TLRs with an agent having the chemical moiety.

Compounds of the Invention

In one embodiment, provided are compounds having the following formulas (I-III):

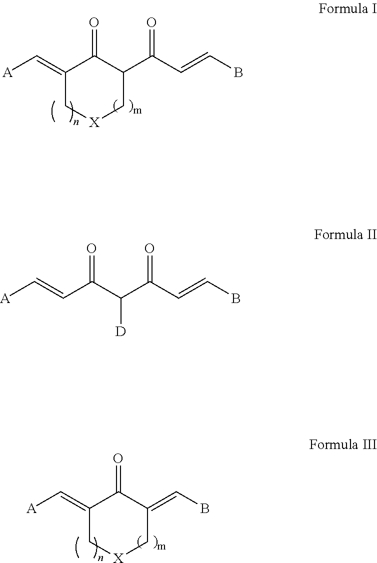

wherein A and B are independently selected from optionally substituted aryl, optionally substituted heterocycle, optionally substituted heteroaryl, or optionally substituted alkyl, wherein A and B are optionally substituted with from 1-5 R groups selected from the group consisting of hydrogen, (C1-C6)alkyl, (C1-C6) alkenyl, (C1-C6) alkynyl, heteroalkyl, halo, (C1-C6) alkoxy, amino, (C1-C6) alkylamino, hydroxy, cyano, nitro, an amino-acid, 5- or 6-member optionally substituted unsaturated, partially unsaturated or saturated heterocyclyl or carbocyclyl optionally substituted with acyl, halo, lower acyl, lower haloakyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;

D is selected from (C1-C6) alkyl, (C1-C6)alkenyl, (C1-C6) alkynyl, heteroalkyl, (C1-C6)alkoxy, amino, (C1-C6)alkylamino, hydroxy, amino acid, optionally substituted unsaturated, partially unsaturated or saturated heterocyclyl or carbocyclyl optionally substituted with acyl, halo, lower acyl, lower haloakyl, oxo, cyano, nitro, carboxyl, amino, amino acid, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;

X is selected from carbon, oxygen, nitrogen, —(CO)N—, —N(CO)—, —(CO)O—, and —O(CO)—, wherein carbon, nitrogen, CON, and NCO are optionally substituted by hydrogen, (C1-C6)alkyl, (C1-C6)alkenyl, (C1-C6)alkynyl, heteroalkyl, (C1-C6)alkoxy, amino, (C1-C6)alkylamino, hydroxyl, amino acid, optionally substituted unsaturated, partially unsaturated or saturated heterocyclyl or carbocyclyl optionally substituted with acyl, halo, lower acyl, lower haloakyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;

n and m are independently 0, 1, 2, wherein n+m are optionally one or more;

or a tautomer, pharmaceutically acceptable salt, ester and/or prodrug thereof.

In some embodiments, A and B are aryl or heteroaryl substituted by R1, R2, R3, R4, and R5, wherein R1, R2, R3, R4, R5 are independently selected from hydrogen, Cl, Br, I, —OR₆, an amino acid attached through its amino or acid function, —OC(O)R₆, OC(O)NR₇R₈, —C(O)R₉, —CN, —NR₁₀R₁₁, —SR₁₂, —S(O)R₁₁, —S(O)₂R₁₄, —C(O)OR₁₅, —S(O)₂NR₁₆R₁₇, and —R₁₈NR₁₉R₂₀ wherein R₆, R₇, R₈, R₉, R₁₀, R₁₁, R₁₂, R₁₃, R₁₄, R₁₅, R₁₆, R₁₇, R₁₈, R₁₉, and R₂₀ are the same or different and are selected from hydrogen or branched or unbranched alkyl groups comprising one to eight carbon atoms optionally linked to each other to form an additional ring. A and B may be phenyls.

In some embodiments, R1, R2, R3, R4, R5 are each hydrogen.

In yet other embodiments, 1-3 of R1, R2, R3, R4, R5 are a heterocyclic or carbocyclic ring, for example a 5- or 6-membered optionally substituted heterocyclic or carbocyclic ring. In certain embodiments, R1, R2, R3, R4, R5 can be an optionally substituted 5-membered ring having one or two heteroatoms selected from O, N and S. In specific embodiments, the heteroatom is selected from O or S.

In yet still further embodiments, R1, R2, R3, R4, R5 can be a 6-membered heterocyclic or carbocyclic ring. In certain embodiments, R1, R2, R3, R4, R5 can be optionally substituted 6-membered ring having one heteroatom selected from O, N and S. In certain embodiments, R₁, R₂, R₃, and R₄ are each optionally substituted 6-membered ring having two heteroatoms selected from O, N, and S.

In yet other embodiments, one or more (1-2. 3, 4 or 5) of R1, R2, R3, R4, R5 is an optionally substituted D- or L-amino acid or an acylated, esterified or amidated form thereof, or a salt thereof. In some embodiments, the amino acid is valine or lysine. In some embodiments, the amino acid is attached through its amine function.

In yet another embodiment, X is a carbon and n=1 and m=1.

In another embodiment, the compounds are selected from the compounds shown in the examples. Other specific embodiments of interest are described in the claims.

Methods of the Invention

The methods described herein are based in part on the applicants' discovery that the presence of the human MGAT3, VDR and/or TLR genes and the corresponding gene product enzyme or receptor activity is predictive of the efficacy of CNS (e.g., anti-AD or anti-ALS) drugs. These methods are also useful for the pre-clinical development of drugs for treating CNS disorders, as well as for conducting clinical trials of drugs for treatment of these diseases and the underlying biological abnormalities.

We suggest that the key problem in AD and ALS lies specifically in the dysfunction of monocytes (macrophages and microglia) of the innate immune system. Heterogeneous defects in macrophage differentiation in vitro, abnormal Aβ uptake and trafficking to lysosomes, and apoptosis on exposure to Aβ has been observed. In addition, patients' monocytes over-express IL-12 and patients' CD4 T cells over produce IL-10 and interferon-gamma, the cytokines belonging to both T_{H}1 and T_{H}2 sets. Recent data from ALS patient macrophages suggest similar defects in cytokine expression. Thus, the adaptive and innate immune system components of AD and ALS patients appear to be in various stages of disharmony and dysfunction. In contrast, macrophages of age-matched control subjects voraciously ingest Aβ and seem to degrade it. The whole innate immune system in AD and ALS patients may be defective and its pathological state can be evaluated by studying peripheral human monocyte/macrophages, genetic markers, and enzyme and receptor activities.

In one embodiment, provided are methods for treatment of AD or ALS comprising administering to a subject in need of such treatment a curcumin or curcumin analog having formulas (I-III).

In another embodiment, methods are particularly useful for determining therapeutic efficacy and/or reducing toxicity, in individuals suffering from a wide number of CNS diseases, quickly and efficiently.

It may be that certain variants of MGAT3, VDR or TLRs are markers for more efficacious AD therapy. Testing new drugs in populations of individuals suffering from AD or ALS, behavioral disorders or other CNS conditions that encoded variants of human MGAT3, VDR or TLRs could provide substantial improvement in therapeutic efficacy and drug discovery. The MGAT3, VDR or TLRs present in recombinant preparations are also useful in in vitro methods to identify drug candidates that are up-regulators for MGAT3, VDR or TLRs that possess superior pharmacological or pharmaceutical properties useful in drug discovery and AD drug development. Thus, screening for MGAT3, VDR or TLR inducers or modulators provides important information as to how to modify the drug candidate to make a drug having a greater therapeutic index and/or decreased toxicity. Human MGAT3, VDR or TLR variants are also useful as a chemical or drug discovery agent in its own right as a means of identifying more highly efficacious drugs.

Further, the present invention relates to using the amino acid differences of human MGAT3, VDR or TLRs to identify new human MGAT3, VDR or TLRs up-regulators that may have superior drug development potential and find use as a bioindicator for drug development in the biotechnology or pharmaceutical industry.

In one embodiment, the present invention provides a method for predicting in an individual the efficacy of a drug, where the drug is an MGAT, VDR or TLRs up-regulator or modulator and the individual is suffering from or at risk of developing a CNS disorder amenable to treatment with the drug. The method generally comprises (1) isolating a biological sample from an individual, where the biological sample includes nucleic acids and/or cellular proteins, and (2) analyzing the biological sample to determine in the individual the presence or absence of the MGAT3, VDR or TLR gene and/or protein. A determination of the presence of the MGAT3, VDR or TLR gene level or enzyme activity is indicative of a positive clinical outcome with administration of the drug for treating the CNS disorder.

In certain embodiments, where the biological sample includes cellular proteins from a tissue that expresses the MGAT3, VDR or TLR genes, the MGAT3, VDR or TLR protein in the sample is analyzed for the presence of the MGAT3, VDR or TLR activity. For example, the determination of the presence in a sample of MGAT3, VDR or TLRs can be carried out as an immunoassay in which the sample is contacted with antibodies capable of binding the MGAT3, VDR or TLR protein. Antibodies (e.g., monoclonal antibodies) can be raised that specifically distinguish between wild-type MGAT3, VDR or TLRs and any MGAT3, VDR or TLR variant. Methods for making antibodies are well-known in the art and are described in, e.g., Harlow and Lane, Antibodies: A Laboratory Manual (Cold Spring Harbor Laboratory, Cold Spring Harbor, N.Y. 1988).

In certain embodiments, the biological sample includes nucleic acids and the sample is analyzed to determine the nucleotide present at positions of codons of the MGAT3, VDR or TLR genes (corresponding to nucleotide positions of SEQ ID NOs:1-12 shown below).

DNA Gene Sequences encoding mRNAs for human MGAT3, TLRs, VDR are identified below by their NCBI Genbank nucleotide accession numbers. Single nucleotide polymorphisms (SNPs) present within the corresponding gene regions can be used to predict disease susceptibility or drug response can be found at the provided URLs for the NCBI public SNP database (dbSNP) based on NCBI genome build 36.3 dated Aug. 3, 2009.

MGAT3 (SEQ ID NO:1)

mRNA acc. no. NM_{—}002409

http://www.ncbi.nlm.nih.gov/nuccore/NM_{—}002409.4?report=genbank

dbSNP: http://www.ncbi.nlm.nih.gov/projects/SNP/snp_ref.cgi?chooseRs=all&go=Go&locusId=4248

TLR1 (SEQ ID NO:2)

mRNA acc. no. NM_{—}003263

http://www.ncbi.nlm.nih.gov/nuccore/41350336

dbSNP: http://www.ncbi.nlm.nih.gov/projects/SNP/snp_ref.cgi?chooseRs=all&go=Go&locusId=7096

TLR 2 (SEQ ID NO:3)

mRNA acc. no. NM_{—}003264

http://www.ncbi.nlm.nih.gov/nuccore/68160956

dbSNP: http://www.ncbi.nlm.nih.gov/projects/SNP/snp_ref.cgi?chooseRs=all&go=Go&locusId=7097

TLR 3 (SEQ ID NO:4)

mRNA acc. no. NM_{—}003265

http://www.ncbi.nlm.nih.gov/nuccore/19718735

dbSNP: http://www.ncbi.nlm.nih.gov/projects/SNP/snp_ref.cgi?chooseRs=all&go=Go&locusId=7098

TLR4 (SEQ ID NO:5)

mRNA acc. no. NM_{—}138554

http://www.ncbi.nlm.nih.gov/nuccore/207028620

dbSNP: http://www.ncbi.nlm.nih.gov/projects/SNP/snp_ref.cgi?chooseRs=all&go=Go&locusId=7099

TLR5 (SEQ ID NO:6)

mRNA acc. no. NM_{—}003268

http://www.ncbi.nlm.nih.gov/nuccore/124248535

dbSNP: http://www.ncbi.nlm.nih.gov/projects/SNP/snp_ref.cgi?chooseRs=all&go=Go&locusId=7100

TLR6 (SEQ ID NO:7)

mRNA acc. no. NM_{—}006068

http://www.ncbi.nlm.nih.gov/nuccore/262527233

dbSNP: http://www.ncbi.nlm.nih.gov/projects/SNP/snp_ref.cgi?chooseRs=all&go=Go&locusId=10333

TLR7 (SEQ ID NO:8)

mRNA acc. no. NM_{—}016562

http://www.ncbi.nlm.nih.gov/nuccore/67944638

dbSNP: http://www.ncbi.nlm.nih.gov/projects/SNP/snp_ref.cgi?chooseRs=all&go=Go&locusId=51284

TLR8 (SEQ ID NO:9)

mRNA acc. no. NM_{—}138636

http://www.ncbi.nlm.nih.gov/nuccore/257196253

dbSNP: http://www.ncbi.nlm.nih.gov/projects/SNP/snp_ref.cgi?chooseRs=all&go=Go&locusId=51311

TLR9 (SEQ ID NO:10)

mRNA acc. no. NM_{—}017442

http://www.ncbi.nlm.nih.gov/nuccore/20302169 dbSNP: http://www.ncbi.nlm.nih.gov/projects/SNP/snp_ref.cgi?chooseRs=all&go=Go&locusId=54106

TLR10 (SEQ ID NO:11)

mRNA acc. no. NM_{—}030956

http://www.ncbi.nlm.nih.gov/nuccore/62865617

dbSNP: http://www.ncbi.nlm.nih.gov/projects/SNP/snp_ref.cgi?chooseRs=all&go=Go&locusId=81793

VDR (SEQ ID NO:12)

mRNA acc. no. NM_{—}001017535

http://www.ncbi.nlm.nih.gov/nuccore/63054844

dbSNP: http://www.ncbi.nlm.nih.gov/projects/SNP/snp_ref.cgi?chooseRs=all&go=Go&locusId=7421

The method can further include determining the genotype of the individual with respect to other MGAT3, VDR or TLR alleles.

In some embodiments, the determination is carried out by analyzing DNA according to well known methods, which include, for example, direct DNA sequencing of the wild-type MGAT3, VDR or TLRs gene, allele specific amplification using the polymerase chain reaction (PCR) enabling detection of either wild-type or variant MGAT3 or TLR sequences, or by indirect detection of the wild-type or variant MGAT3, VDR or TLR genes by various molecular biology methods including, e.g., PCR-single stranded conformation polymorphism (SSCP)-method or denaturing gradient gel electrophoresis (DGGE). Determination of the wild-type or variant MGAT3, VDR or TLR genes can also be done by using the restriction fragment length polymorphism (RFLP)-method, which is particularly suitable for genotyping large number of samples. As used herein, “wild-type MGAT3, VDR or TLR genes” refers to an allele of the MGAT3, VDR or TLR genes that (a) encodes a gene product that performs the normal function of MGAT3, VDR or TLRs and (b) does not contain MGAT3, VDR or TLRs mutations.

The determination can also be carried out at the level of RNA by analyzing RNA expressed in the sample using various methods. Allele specific probes can be designed for hybridization. Hybridization can be done using, e.g., Northern blot, RNase protection assay, or in situ hybridization methods. RNA derived forms of the wild-type or variant MGAT3, VDR or TLR genes can also be analyzed by converting isolated tissue RNA first to cDNA and thereafter amplifying cDNA by an allele specific PCR-method and carrying out the analysis as for genomic DNA as mentioned above.

Particularly suitable methods for analyzing the nucleic acids include hybridization between the nucleic acid sample and an MGAT3, VDR or TLR nucleic acid probe or primer specific for the wild-type or variant MGAT3, VDR or TLR alleles. Accordingly, nucleic acid molecules particularly useful in accordance with the methods provided herein are oligonucleotides capable of hybridizing, under stringent hybridization conditions, with complementary regions of the MGAT3, VDR or TLRs gene that include the site associated with any MGAT3, VDR or TLR mutation.

A nucleic acid can be DNA or RNA, and single- or double-stranded. Oligonucleotides can be naturally occurring or synthetic, but are typically prepared by synthetic means. Oligonucleotides of the invention include segments of DNA, or their complements, corresponding to the human MGAT3, VDR or TLR genes and including the nucleotide at position of key codons (corresponding to nucleotide positions in SEQ ID Nos:1-12, and/or a base adjacent thereto, of either the variant or wild-type allele. The segments are usually between 5 and 100 contiguous bases, and often range from 5, 10, 12, 15, 20, or 25 nucleotides to 10, 15, 30, 25, 20, 50 or 100 nucleotides. Nucleic acids between 5-10, 5-20, 10-20, 12-30, 15-30, 10-50, 20-50, or 20-100 bases are common

Oligonucleotides of the present invention can be RNA, DNA, or derivatives of either. The minimum size of such oligonucleotides is the size required for formation of a stable hybrid between the oligonucleotide and a complementary sequence on a nucleic acid molecule corresponding to the human MGAT3, VDR or TLRs genes. The present invention includes oligonucleotides that can be used as, for example, probes to identify nucleic acid molecules or primers to produce nucleic acid molecules. Also provided are oligonucleotides that can be used as primers to amplify DNA.

In some embodiments, the oligonucleotide probes or primers include single base change of a MGAT3, VDR or TLR polymorphism (positions of key codons) or the wild-type nucleotide that is located at the same position. The single base change or corresponding wild-type nucleotide can occur within any position of the oligonucleotide. Preferably the nucleotide of interest occupies a central position of a probe. In certain embodiments, the nucleotide of interest occupies a 3′ position of a primer.

Polymorphisms are detected in a target nucleic acid from an individual being analyzed. For assay of genomic DNA, virtually any biological sample (other than pure red blood cells) is suitable. For example, convenient tissue samples include whole blood, blood cells, semen, saliva, tears, urine, fecal material, sweat, buccal epithelium, skin and hair. For assay of cDNA or mRNA, the tissue sample must be obtained from an organ in which the target nucleic acid is expressed.

Methods described below require amplification of DNA from target samples. This can be accomplished by, e.g., PCR. See generally, e.g., PCR Technology: Principles and Applications for DNA Amplification (H. A. Erlich ed., Freeman Press, NY, N.Y., 1992); PCR Protocols: A Guide to Methods and Applications (Innis et al. eds., Academic Press, San Diego, Calif., 1990); Mattila et al., Nucleic Acids Res. 19, 4967 (1991); Eckert et al., PCR Methods and Applications 1, 17 (1991); PCR (McPherson et al. eds., IRL Press, Oxford); and U.S. Pat. No. 4,683,202.

Other suitable amplification methods include the ligase chain reaction (LCR) (see, e.g., Wu and Wallace, Genomics 4,560 (1989), Landegren et al., Science 241, 1077 (1988)), transcription amplification (see, e.g., Kwoh et al., Proc. Natl. Acad. Sci. USA 86, 1173 (1989)), self-sustained sequence replication (see, e.g., Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The identity of the base occupying a polymorphic site at key codons of the MGAT3, VDR or TLR genes can be determined in an individual by several methods.

Single Base Extension Methods:

Single base extension methods are described by, e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to, but does not span a site of, potential variation in the target sequence. That is, the primer comprises a subsequence from the complement of a target polynucleotide terminating at the base that is immediately adjacent and 5′ to a polymorphic site. The term primer refers to a single-stranded oligonucleotide capable of acting as a point of initiation of template-directed DNA synthesis under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer but typically ranges from 15 to 40 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The hybridization is performed in the presence of one or more labeled nucleotides complementary to base(s) that may occupy the site of potential variation. For example, for biallelic polymorphisms, two differentially labeled nucleotides can be used. For tetra allelic polymorphisms, four differentially-labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation if the target sequence is present. Extension incorporates a labeled nucleotide thereby generating a labeled extended primer. If multiple differentially-labeled nucleotides are used and the target is heterozygous then multiple differentially-labeled extended primers can be obtained. Extended primers are detected providing an indication of which base (s) occupy the site of variation in the target polynucleotide.

Allele-Specific Probes:

The design and use of allele-specific probes for analyzing polymorphisms is described by, e.g., Saiki et al., Nature 324, 163-166 (1986); Dattagupta, EP 235,726; Saiki, WO89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent such that there is a significant difference in hybridization intensity between alleles, and preferably an essentially binary response, whereby a probe hybridizes to only one of the alleles. Hybridizations are usually performed under stringent conditions that allow for specific binding between an oligonucleotide and a target DNA containing one the polymorphic site. Stringent conditions are defined as any suitable buffer concentrations and temperatures that allow specific hybridization of the oligonucleotide to highly homologous sequences spanning the MGAT3, VDR or TLRs wild type or polymorphic site and any washing conditions that remove non-specific binding of the oligonucleotide. For example, conditions of 5×SSPE (750 mM NaCl, 50 mM Na Phosphate, 5 mM EDTA, pH 7.4) and a temperature of 23° C. are suitable for allele-specific probe hybridizations. The washing conditions usually range from room temperature to 60° C. Some probes are designed to hybridize to a segment of target DNA such that the polymorphic site aligns with a central position (e.g., in a 15 mer at the 7 position; in a 16 mer, at either the 8 or 9 position) of the probe. This probe design achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence. The polymorphisms can also be identified by hybridization to nucleic acid arrays, some examples of which are described by WO 95/11995.

Allele-Specific Amplification Methods:

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer that hybridizes at a distal site. Amplification proceeds from the two primers leading to a detectable product signifying that the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO93/22456. In other methods, a double-base mismatch is used in which the first mismatch is included in the 3′-most position of the oligonucleotide aligned with the polymorphism and a second mismatch is positioned at the immediately adjacent base (the pen-ultimate 3′ position). This double mismatch further prevents amplification in instances in which there is no match between the 3′ position of the primer and the polymorphism.

Direct-Sequencing:

The direct analysis of the sequence of polymorphisms of the present invention can be accomplished using either the dideoxy-chain termination method or the Maxam Gilbert method (see Sambrook et al., Molecular Cloning, A Laboratory Manual (2nd ed., CSHP, New York 1989); Zyskind et al., Recombinant DNA Laboratory Manual (Acad. Press, 1988)).

Denaturing Gradient Gel Electrophoresis:

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification (W. H. Freeman and Co, New York, 1992), Chapter 7.

Single-Strand Conformation Polymorphism Analysis:

Alleles of target sequences can be differentiated using single-strand conformation polymorphism analysis, which identifies base differences by alteration in electrophoretic migration of single stranded PCR products, as described in Orita et al., Proc. Nat. Acad. Sci. USA 86, 2766-2770 (1989). Amplified PCR products can be generated as described above, and heated or otherwise denatured, to form single stranded amplification products.

Single-stranded nucleic acids may refold or form secondary structures that are partially dependent upon the base sequence. The different electrophoretic mobilities of single stranded amplification products can be related to base-sequence differences between alleles of target sequences.

Once the presence or absence of MGAT3, VDR or TLR wild type or variant allele is determined for an individual, this information can be used in different ways. For example, as set forth above, a determination that the MGAT3, VDR or TLR gene or enzyme is present is indicative of the susceptibility to disease or the efficacy of the drug for the treatment of a CNS disorder (e.g., AD or other neurodegenerative). Thus, the information can be used to help determine an appropriate diagnostic or treatment regimen, respectively, for an individual suffering from the disorder.

Determination of the presence or absence of the MGAT3, VDR or TLR wild type or variant alleles is also useful for conducting clinical trials of drug candidates for CNS disorders. Such trials may be performed on treated or control populations having similar or identical polymorphic profiles at a defined collection of polymorphic sites. Use of genetically matched populations eliminates or reduces variation in treatment outcome due to genetic factors, leading to a more accurate assessment of the efficacy of a potential drug.

Furthermore, the determination of the presence or absence of the MGAT3, VDR or TLR genes or a variant allele may be used after the completion of a clinical trial to elucidate differences in response to a given treatment. For example, the information may be used to stratify the enrolled patients into disease subtypes or classes. It may further be possible to use the methods described herein to identify subsets of patients with similar polymorphic profiles who have unusual (high or low) response to treatment or who do not respond at all (non-responders). In this way, information about the underlying genetic factors influencing response to treatment can be used in many aspects of the development of treatments (these range from the identification of new targets, through the design of new trials to product labeling and patient targeting). Additionally, the methods may be used to identify the genetic factors involved in adverse response to treatment (adverse events). For example, patients who show an adverse response may have a higher incidence of the absence of the MGAT3, VDR or TLR allele than observed in the general population. This would allow the early identification and exclusion of such individuals from treatment. It would also provide information that might be used to understand the biological causes of adverse events and to modify the treatment to avoid such outcomes.

In another aspect, the present invention provides methods for screening for MGAT3, VDR or TLR up-regulation activity using the variant and/or wild-type MGAT3, VDR or TLRs protein. These methods can provide information as to how to modify a drug candidate to make a more efficacious and/or safer drug for the treatment of a CNS disorder such as, e.g., AD or ALS.

In another aspect, the present invention also provides a method to remove blood from an AD or ALS patient, isolate and treat white or other blood cells with an agent that increases MGAT3, VDR and/or TLR activity. After removal of the agent, the cells are returned to the AD or ALS patient for treatment of AD, ALS or other CNS diseases.

In certain embodiments, a predetermined therapeutic agent (e.g., curcumin) for the treatment of a CNS disorder is derivatized to create one or more analog candidate agents. The agent will typically retain one or more moieties associated with therapeutic efficacy, while incorporating one or more moieties that are or known or predicted to be a potential inducer moiety for MGAT3, VDR or TLRs. MGAT3, VDR or TLRs inducer moieties are not generally known but can include, for example, chemical centers such as, e.g., a chemical center analogous to that contained in curcumin.

Methods of chemical modification suitable for use in accordance with the methods provided herein are generally known in the art. For example, an MGAT3, VDR or TLR inducer moiety (e.g., a curcumin group) can be linked to the predetermined therapeutic agent.

The derivatized agent is tested to determine if the agent is an inducer for the MGAT3, VDR or TLR protein. Greater levels of MGAT3 enzyme or VDR or TLR activity in the presence of the derivatized agent relative to the underivatized, predetermined therapeutic agent is generally indicative of greater efficacy and/or lower toxicity of the derivatized agent relative to the underivatized therapeutic agent. In certain embodiments, a library of derivatized agents is screened to identify one or more candidate agents that are inducers for MGAT3, VDR or TLRs. MGAT3, VDR or TLR proteins suitable for use in accordance with these methods include, e.g., wild-type and variant MGAT3, VDR or TLRs.

In one embodiment, a method for predicting the efficacy of a candidate agent for the treatment of a CNS disorder is provided which includes: (1) contacting a wild type sample of an MGAT3, VDR or TLR protein with the candidate agent; (2) contacting a second AD or ALS sample of an MGAT3, VDR or TLR protein with a predetermined therapeutic agent; where the contacting of each of the first and second samples is under conditions suitable for supporting MGAT3 enzyme or VDR or TLR activity; (3) determining for each of the first and second samples the level of MGAT3 enzyme or VDR or TLR activity; and (4) comparing the level of MGAT3 enzyme or VDR or TLR activity in the first sample with the level of MGAT3 enzyme or VDR or TLR activity in the second sample. A greater level of MGAT3 enzyme or VDR or TLR activity in the second sample relative to the first sample is indicative of efficacy of the candidate agent for treatment of the disorder. In certain embodiments, the predetermined therapeutic agent is an anti-AD or ALS drug such as, e.g., curcumin or some other immune modulator. Particularly suitable are candidate agents having a curcumin center analogous to the center of curcumin.

The MGAT3, VDR or TLR protein sample can include, e.g., a sample comprising a recombinant form of the protein in a cellular or a cell-free preparation. Methods for producing and isolating catalytically active, recombinant human MGAT3, VDR or TLR protein are known in the art. (See, e.g., Bhattacharyya et al., J. Biol. Chem. 277:26300-26309 (2002).

MGAT3 or TLR protein suitable for use in accordance with the present methods can also be obtained from tissues or cells that express the MGAT3, VDR or TLR protein endogenously. For example, tissues or cells expressing MGAT3, VDR or TLR protein may be use to prepare enzyme for use in MGAT3, VDR or TLR enzyme activity assays. Monocytes, kidney or brain cells are a particularly suitable source for MGAT3, VDR or TLR proteins. Kidney or brain samples suitable for use in the preparation of enzyme can be obtained from banks of cryopreserved human or mouse tissue. Methods for preparing human or mouse kidney or brain containing viable MGAT3, VDR or TLR protein, and as well as method for using enzyme assays in MGAT3, VDR or TLR activity assays, are known. (See, e.g., e.g., Bhattacharyya et al., J. Biol. Chem. 277:26300-26309 (2002)). In certain embodiments, tissues or cells used for preparation of MGAT3, VDR or TLR protein are homozygous for either variant or wild-type MGAT3, VDR or TLR. In other embodiments, a protein sample containing variant MGAT3, VDR or TLR is derived from tissue or cells heterozygous for a variant allele.

In certain embodiments, the sample comprises cells, cultured in vitro, expressing MGAT3, VDR or TLR. The cells can express either recombinant or endogenous MGAT3, VDR or TLR protein. Particularly suitable cells for endogenous expression of MGAT3, VDR or TLR include human kidney cells or transfected CHO cells or human or animal monocytes. Cells expressing an endogenous variant MGAT3, VDR or TLR alleles can be either homozygous or heterozygous. With respect to recombinant cells, methods for cloning genes encoding the MGAT3, VDR or TLR protein, production of recombinant expression vectors, transfection of cells, and subsequent expression of the encoded protein are known in the art. (See generally, e.g., Sambrook and Russell, Molecular Cloning, A Laboratory Manual (3rd ed. 2001); Ausubel et al. (eds.), Current Protocols in Molecular Biology (1994); Sambrook et al., Molecular Cloning, A Laboratory Manual (2nd ed. 1989).) Methods for determining MGAT3, VDR or TLR activity in cultured cells are also generally known in the art. (See, e.g., Bhattacharyya et al., J. Biol. Chem. 277:26300-26309 (2002)).

Suitable methods for determining the level of MGAT3, VDR (or TLR) enzyme activity typically include, for example, detection of N-glycosylation associated with MGAT3 enzyme activity or binding to TLR. For MGAT3, a particularly suitable assay is the detection of an N-glycosylation of a peptide or a protein (See, e.g., Bhaumik et al., Cancer Res. 58, 2881-2887). For example, the method can include detection of a N-glycosylated peptides or proteins.

Methods of sample preparation and product identification, including identification of N-glycosylated products, are well-known in the art and include, for example, the use of HPLC methods (e.g., reverse HPLC-tandem mass spectrometry (HPLC-MS/MS) or TLC methods). (See, e.g., Bhaumik et al., Cancer Res. 58, 2881-2887).

Ex Vivo Therapy of Alzheimer's Disease or ALS

In another embodiment, provided is a method for ex vivo therapy for patients with AD or ALS. This method comprises the steps of obtaining a blood sample from an AD or ALS patient, contacting the blood sample with the compounds of the invention, and injecting the treated blood sample back into the AD or ALS patient.

Pharmaceutical Compositions of the Invention:

Provided herein are pharmaceutical compositions comprising one or more compounds of Formulas I-III as active ingredients or a pharmaceutically acceptable salt, solvate, or prodrug thereof, in a pharmaceutically acceptable vehicle, carrier, diluent, or excipient, or a mixture thereof.

Provided herein are pharmaceutical compositions in modified release dosage forms, which comprise one or more compounds of Formulas I-III or a pharmaceutically acceptable salt, solvate, or prodrug thereof; and one or more release controlling excipients as described herein.

Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further provided herein are pharmaceutical compositions in enteric coated dosage forms, which comprise one or more compounds of Formulas I-III or a pharmaceutically acceptable salt, solvate, or prodrug thereof; and one or more release controlling excipients for use in an enteric coated dosage form. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours.

In one embodiment, the pharmaceutical compositions comprise one or more compounds of Formulas I-III or a pharmaceutically acceptable salt, solvate, or prodrug thereof; and one or more release controlling and non-release controlling excipients, such as those excipients suitable for a disruptable semi-permeable membrane and as swellable substances.

Provided herein are pharmaceutical compositions that comprise about 0.1 to about 100 mg, about 0.5 to about 75 mg, about 1.0 to about 50 mg, about 2.5 to about 25.0 mg, about 5.0 to about 15 mg, about 0.1 mg, about 0.5 mg, about 1 mg, about 5 mg or about 10 mg, of one or more compounds of Formula I-III as a sterile solution for injection per day. The pharmaceutical compositions further comprise about 0.1% to about 2% sodium chloride, about 0.1% to about 2% ammonium acetate, about 0.001% to about 0.1% edetate disodium, about 0.1% to about 2% benzyl alcohol, with a pH of about 6 to about 8.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary depending on a condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Routes of Administration:

Depending on the condition, disorder, or disease, to be treated and the subject's condition, a compound provided herein may be administered by oral, parenteral (e.g., intramuscular, intraperitoneal, intravenous, ICV, intracistemal injection or infusion, subcutaneous injection, or implant), inhalation, nasal, vaginal, rectal, or sublingual routes of administration, and may be formulated, alone or together, in suitable dosage unit with pharmaceutically acceptable carriers, adjuvants and vehicles appropriate for each route of administration.

Parenteral Administration:

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

Controlled-Release Dosage Forms:

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiment, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

Dosing:

In certain embodiments, provided compounds are administered once daily in a single or divided dose in the amount of about 0.1 to about 100 mg/kg per day for parenteral administration, where kg refers to a subject's body weight.

In certain embodiments, provided compounds are administered once daily in a single or divided dose in the amount of about 0.5 to about 75 mg/kg per day.

In certain embodiments, provided compounds are administered once daily in a single or divided dose in the amount of about 1.0 to about 50 mg/kg per day.

In certain embodiments, provided compounds are administered once daily in a single or divided dose in the amount of about 2.5 to about 25.0 mg per day.

In certain embodiments, provided compounds are administered once daily in a single or divided dose in the amount of about 5.0 to about 15 mg per day.

In certain embodiments, provided compounds are administered once daily in a single or divided dose in the amount of about 0.1 mg, about 0.5 mg, about 1 mg, about 5 mg or about 10 mg of one or more compounds of Formula I-III for parenteral administration per day.

The invention is further described by the following non-limiting examples.

### EXAMPLES

Curcumins enhance expression of genes associated with Aβ phagocytosis. An in vitro system using cultured monocytic cell lines has been developed for rapidly evaluating novel curcumin analogs for potency and for improved pharmaceutical properties. A fingerprint profile of gene expression in response to a new chemical entity could provide insight as a drug development candidate. These studies complement ex vivo and in vivo studies of curcumin-enhanced removal of amyloid from brain sections.

### Example 1

Cell culture conditions for monocytic cell lines. Human and mouse monocyte cell lines are grown in RPMI-1640 medium with 10% fetal bovine serum (FBS), plus glutamine and pen/strep. For treatment with compounds (curcuminoids+/−Aβ), replicate sets of 0.5-1.0 mL cultures containing 1-2×10⁶ cells are set up in the wells of 48-well tissue culture plates. Both replicate sets have at least one control well receiving no added curcuminoid to provide baseline expression controls for untreated, and Aβ-only treated cells. Remaining wells receive curcuminoid in a range of 10-fold serial dilutions in DMSO, typically from 0.1 nM-1 Cultures are incubated for 20-24 hours (“overnight”) at 37° C. in atmosphere with 5% CO₂. After one overnight, to one of replicate sets of cultures is added Aβ (1-42) peptide to a final concentration of 5 μg/mL.

RNA isolation and cDNA synthesis. After the first overnight incubation in the presence of curcuminoid, cells from one replicate set of wells are collected by centifugation. RNAs are extracted from pelleted cells using miniprep column kits following manufacturer's protocol for animal cells. One mL of total cellular RNA from each well is quantitated using a Nanodrop spectrophotometer, and first-strand total cDNA is prepared from 0.3-1.0 μg RNA by random-hexamer priming. Following the second 20 hr. incubation in the presence of Aβ, RNA extractions and cDNA synthesis are similarly performed.

### Example 2

### Gene Expression Using Quantitative, Real-Time PCR (qPCR)

Assay development. Gene-specific mRNA levels are determined using a real-time thermocycler, and by normalizing to expression of one or more control genes. Assays for quantifying mRNA levels are based on gene sequences reported in NCBI Genbank. PCR oligonucleotide primers are selected spanning introns to minimize the opportunity for amplication from any contaminating genomic DNA in the RNA preps. Oligonucleotides are typically tested and used at a concentration of 200 nM in qPCR reactions containing SYBR dye. Control reactions include template from cDNA synthesis reactions either + or − reverse transcriptase (RT), as well as no-template controls (ntc). All reactions are performed in triplicate. Reactions are typically run with 40 cycles of 95° C. (10 sec) and 58° C. (30 sec). Melt curves based on incremental 0.5° C. increases are determined for new assays to test for uniformity of amplified products. Reaction conditions are modified as needed depending on initial test results. ‘Working’ assays are selected based on clear RNA-dependent amplification and absence of product in ‘ntc’ reactions. The following assay primers are used:

| MGAT3 (human), | |
|---|---|
| 5′-CGGACGCCAGCATCTCC; | (SEQ ID NO. 13) |
| 3′-AGGGCTGAGGGAGGCCAGTTCTC; | (SEQ ID NO. 14) |
| MGAT3 (mouse), | |
| 5′-CGGACGATGGGATGAAGATG; | (SEQ ID NO. 15) |
| 3′-AGGCCAGTTCTCTCGGGAAG; | (SEQ ID NO. 16) |
| TLR2 (human), | |
| 5′-GCGTTCTCTCAGGTGACTGC; | (SEQ ID NO. 17) |
| 3′-CCCACAGGTACCTTCACTTGG; | (SEQ ID NO. 18) |
| TLR2 (mouse), | |
| 5′-TTCAGTCTTCCTAGGCTGGTG; | (SEQ ID NO. 19) |
| 3′-ACACATCTCCTGCCAGTGAC; | (SEQ ID NO. 20) |
| TLR3 (human), | |
| 5′-AAAGGAAAGGCTAGCAGTCATCC; | (SEQ ID NO. 21) |
| 3′-CAGTGCACTTGGTGGTGGAG; | (SEQ ID NO. 22) |
| TLR3 (mouse), | |
| 5′-TCATTTCGTTATCACACACCA; | (SEQ ID NO. 23) |
| 3′-AATTGAATCCAGATTTTGCTCA; | (SEQ ID NO. 24) |
| TLR4 (human), | |
| 5′-ATCCCCTGAGGCATTTAGGC; | (SEQ ID NO. 25) |
| 3′-TGCCCCATCTTCAATTGTCTG; | (SEQ ID NO. 26) |
| TLR4 (mouse), | |
| 5′-GCTCCTGGCTAGGACTCTGA; | (SEQ ID NO. 27) |
| 3′-TGTCATCAGGGACTTTGCTG; | (SEQ ID NO. 28) |
| VDR (human), | |
| 5′-CAGCATCCAAAAGGTCATTGG; | (SEQ ID NO. 29) |
| 3′-CAATGGCACTTGACTTCAGCAG; | (SEQ ID NO. 30) |
| VDR (mouse), | |
| 5′-CCAAAAGGTCATCGGCTTTG; | (SEQ ID NO. 31) |
| 3′-GGAGCGCAACATGATCACC; | (SEQ ID NO. 32) |

qPCR reactions of cultured monocyte cDNAs. One μL total cellular cDNA per reaction is used in triplicate reactions for each test gene, and for each culture treatment. SYBR-Green reactions were run on a Continuous Fluorescence real-time thermocycler and analyzed. Cycle threshold (Ct) values are recorded for each replicate reaction, and are then averaged and normalized against mean Ct values from one or more control genes. Fold-expression differences relative to untreated cells are determined using the 2^{-ΔΔCt} method as previously described. Data from the curcumin concentration range are plotted in order to determine EC₅₀ values (concentrations producing one-half maximal stimulant activity), which are used for comparisons of potency between different compounds. Data are also evaluated for each gene with regard to sensitivity, maximally effective concentration of curcuminoid, response to Aβ, and overall magnitude of fold-expression changes.

Data showing in vitro up-regulation of genes associated with Aβ phagocytosis in response to curcumins. Natural curcumins and synthetic analogs were tested in U-937 human monocyte cells in vitro for their ability to modulate expression of the genes MGAT3 and TLRs −2, −3, and −4. Levels of mRNAs transcribed by these genes were analyzed by quantitative PCR (qPCR) after an overnight (24-hour) incubation in varying concentrations of curcumin, followed by another overnight incubation with Aβ peptide at 5 μg/mL. In addition, given that 1,25(OH)2D3, the biologically active form of vitamin D3, has been shown to stimulate monocytic differentiation to phagocytic macrophages, we also examined response of the vitamin D3 system to curcumins by testing expression of the vitamin D receptor (VDR) gene in similarly treated cells. After treatments, cultured cells were lysed, total cellular RNAs extracted, and total cDNAs prepared as described above. Control cultures included cells receiving either no curcumin treatment, or Aβ-only. Expression data were normalized to those of the control gene, HPRT.

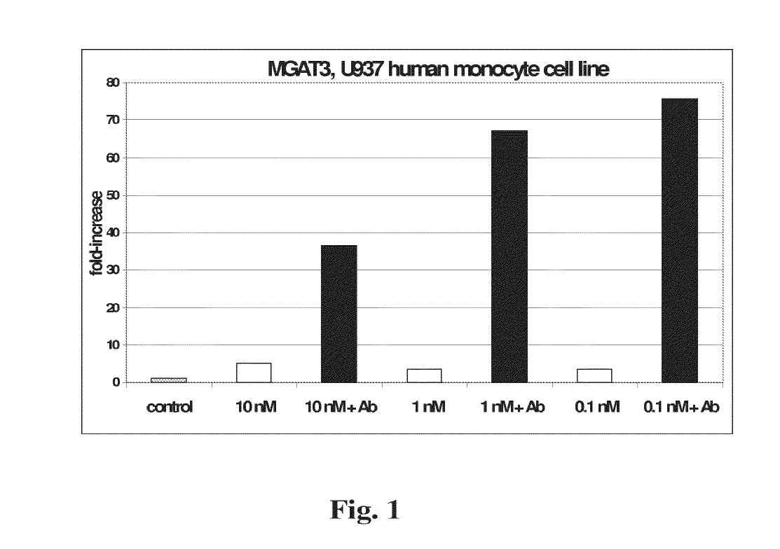

Data are presented from testing responses in MGAT3, TLR, and VDR gene expression to the natural curcuminoid BDC (compound 1), synthetic water-soluble versions of BDC and Curcumin (compounds 33 and 36, respectively), and compound 10 (see Example 8, Synthesis of novel curcumin analogs). FIG. 1 shows normalized MGAT3 gene expression data presented as fold increases in gene-specific mRNA levels in treated cells relative to untreated controls, in response to varying concentrations of compound 33 and to Aβ treatment. As for most tested genes (see following data) the greatest responses in MGAT3 expression were observed for curcumins in the range of 0.1-10 nM. Curcumin treatment alone had relatively little effect on MGAT3 mRNA levels, however curcumins potentiated gene expression increases upon subsequent exposure to Aβ in a dose-dependent manner. The relative responses of all tested genes to each curcumin plus Aβ, including MGAT3, are summarized in Table 2.

### Example 3

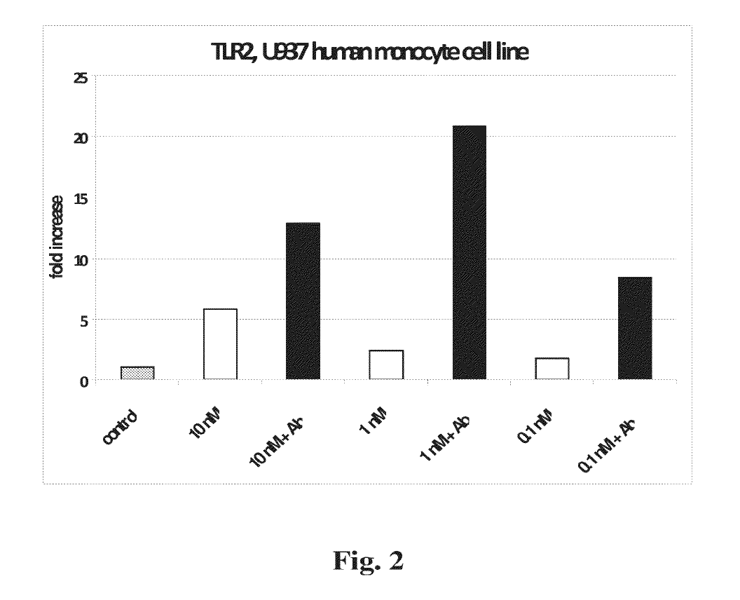

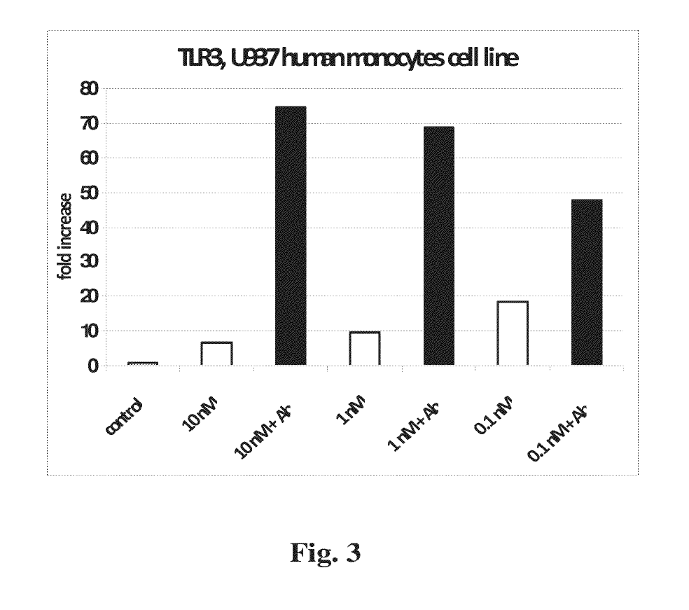

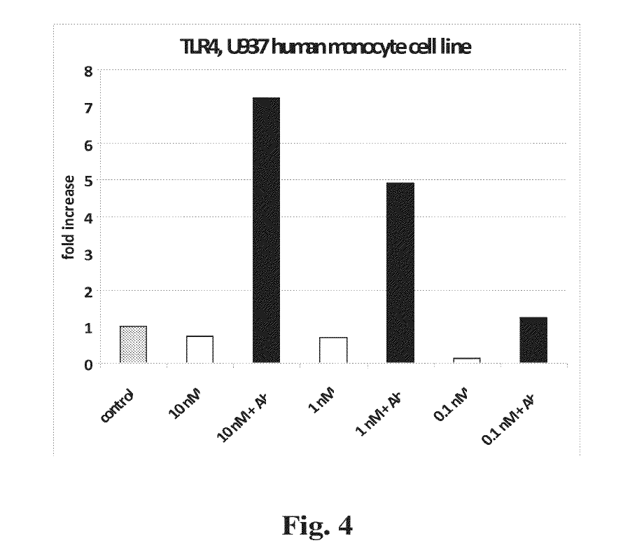

TLR gene expression. Expression of genes encoding Toll-like receptors −2, −3 and −4 was similarly tested in response to curcumins and to Aβ (FIGS. 2-4). Compared to MGAT3, normalized expression of TLR2 and TLR4 was relatively less responsive to the tested curcumins, with natural BDC (compound 1) and synthetic analogs inducing lower increases in expression. TLR2 showed slightly higher expression in response to compound 10, with maximal expression at 1 nM concentration in the presence of Aβ (FIG. 2). TLR3 showed limited response to BDC (1), and compounds 33 and 36, plus Aβ. However this gene showed considerably higher expression in response to compound 10, at all concentrations tested (FIG. 3). TLR4 showed modest expression increases in response to all four tested curcumins, with maximal response at approximately 10 nM compound 10 (FIG. 4).

In addition to relative differences in maximal response, the different TLR genes tested showed independent profiles with regard to curcumin concentrations inducing the greatest response. These profiles, individually and together with other genes, have utility for evaluating novel curcumins as AD and ALS drug candidates.

### Example 4

### VDR Gene Expression

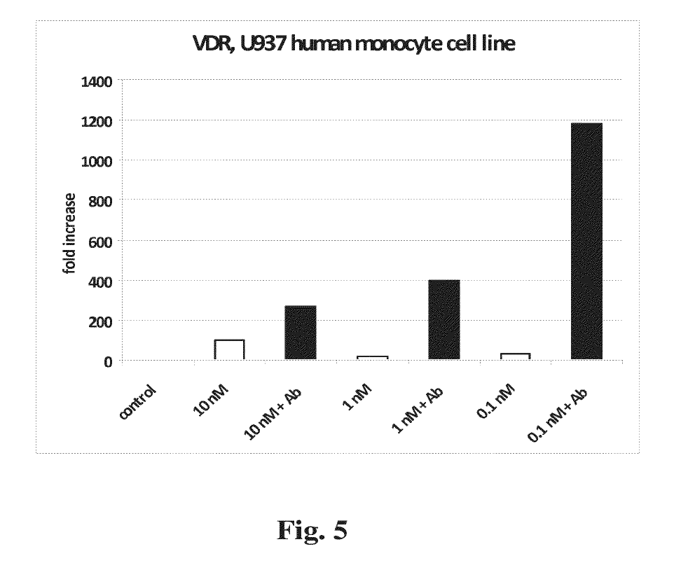

Given that 1,25(OH)2D3, the biologically active form of vitamin D3, has been shown to stimulate monocytic differentiation to phagocytic macrophages, we also examined response of the vitamin D3 system to curcumins by testing expression of the vitamin D receptor (VDR) gene. In our cell culture system using human monocytes, this gene showed modest increases in normalized expression in response to compounds BDC (1) and 36, and somewhat higher expression in response to 33, plus Aβ. However VDR showed the greatest response (10³-fold increase) to compound 10 plus Aβ (FIG. 5).

Summary: The results of testing expression of MGAT3, TLRs 2-4, and VDR are summarized in Table 2. By examining levels of the same mRNAs, these results allow comparisons between different curcumin analogs for potency in stimulating genes associated with innate immune cell function, with relevance to Aβ removal (Proc Natl Acad Sci USA 104 (2007) 12849). Similarly, the respective genes showed relative differences in sensitivity of their responses to different compounds in both concentration-dependence and maximal response. All genes showed relatively modest expression increases in response to natural BDC (1). This may be due in part to limitations of BDC with regard to solubility and stability. Certain analogs of natural curcumins designed to address these limitations have shown improved potency and drug-like properties. While one water-soluble analog of natural Curcumin (36) induced modest increases in expression of all genes, a water-soluble analog of BDC (33) induced higher expression of both VDR and especially MGAT3. Compound 10 induced response of most genes, in particular MGAT3, TLR3, and VDR. Thus, in addition to individual responses, certain genes also showed similar response patterns to common curcumins. These results emphasize the value of this in vitro assay system for evaluating structure-activity relationships, and the potential of new curcumin analogs for stimulating expression of markers of innate immune cell function. In advance of in vivo testing, these assays will accelerate further development of optimized curcumin compounds as novel AD and/or ALS drugs.

TABLE 2
Relative gene expression in human monocytic cells.

| | Compound | | | |
|---|---|---|---|---|
| Gene | 1 | 36 | 33 | 10 |
| TLR4 | + | + | + | + |
| TLR2 | + | + | + | ++ |
| MGAT3 | + | + | +++ | +++ |
| TLR3 | + | + | + | +++ |
| VDR | + | + | ++ | +++++ |

Increased expression relative to control cells indicated by: (+) <20-fold; (++) >20-fold; (+++) >40-fold; (++++) >100-fold; (+++++) >1000-fold.

### Example 5

### Gene Expression as Biomarkers of Pathology in Mouse Models of AD

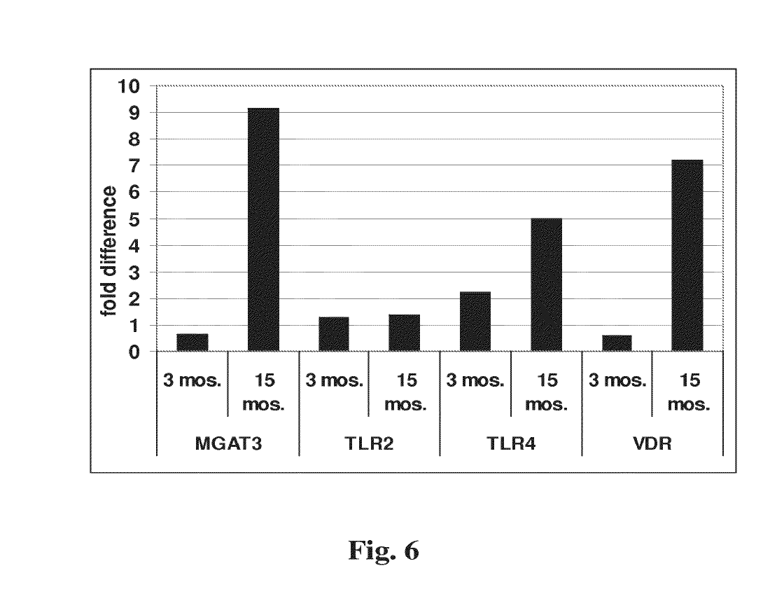

The same genes examined in the in vitro cell culture assay may also represent useful biomarkers for development of neurodegenerative disease in rodent models. Using human genes linked to familial AD, predominantly involving mutant amyloid precursor protein (APP) and presenilin (PS) genes, a number of transgenic mouse strains have been created that manifest age-dependent amyloid pathology resembling AD development in humans. Analysis of in vivo expression of these genes in samples from these mice can lend insights into physiologic changes accompanying onset of CNS pathology, and identify key stages of disease development representing targets for therapeutic intervention to prevent and/or minimize cognitive decline. These mouse models also provide the opportunity for in vivo testing of compounds previously optimized in vitro for efficacy of Aβ removal in vivo. We have obtained samples from two independent transgenic mouse AD models for the purpose of characterizing in vivo mRNA levels of MGAT3, TLRs 2-4, and VDR. These models overexpress different mutant human genes, but both show age-dependent accumulation of amyloid plaque in CNS tissue. Peripheral blood monocytes harvested from whole blood, plus tissues from whole brain, liver, and kidney, were obtained from both mouse models from ages representing pre-(3-4 months) and post-onset of pathology (15-20 months), and from healthy control (nontransgenic) mice. Following extraction of total cellular RNA, synthesized cDNAs were used to measure mRNA levels by qPCR (FIG. 6).

Several genes showed increased expression in peripheral monocytes from aged transgenic mice, relative to aged control mouse monocytes. TLR2 showed similar expression levels in monocytes from both young and aged AD and control mice. The TLR4 and VDR genes showed greater relative increases in AD over control monocytes from aged mice. MGAT3 showed the greatest increase in aged AD monocytes among the tested genes. TLR3 showed low or undetectable expression in these cells. It may be that monocytes in vivo are responsive either to signals resulting from onset of CNS pathology, or to ectopic transgene expression in peripheral tissues. Stimulation of the innate immune system may normally be part of a coordinated response to counter accumulating Aβ deposition. These data may provide insights into human AD pathology, as peripheral monocytes from AD patients are typically deficient in Aβ phagocytosis and show down-regulated expression of MGAT3 and TLRs in response to Aβ. Monocyte expression of these genes may be a novel biomarker for evaluating in vivo efficacy of curcumin compounds as potential AD drugs.

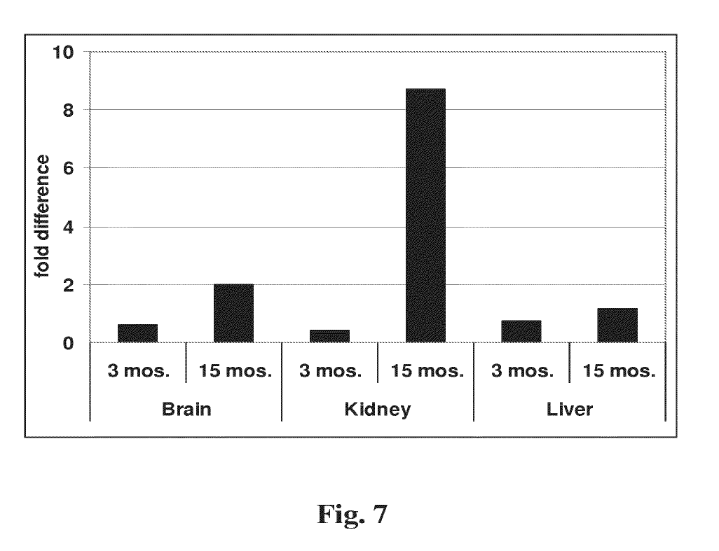

In addition, we examined expression of the gene encoding transthyretin (TTR) in AD model and control mouse samples. This gene is reported to bind soluble Aβ peptide in vitro, and prevents Aβ from forming amyloid fibrils. In the CNS, TTR is expressed predominantly in the choroid plexus, from which the protein is unidirectionally secreted into the cerebrospinal fluid (CSF) where it may have a role in regulating Aβ deposition in vivo. TTR is also expressed in peripheral tissues, predominantly the liver. Shown in FIG. 7, TTR mRNA levels were approximately two-fold higher in aged AD brain relative to aged controls. While this may reflect a physiologic response to counter accumulating Aβ in AD mice, the relatively modest increase may be explained by the very limited expressing cell type within the brain (choroids plexus). However aged AD mice showed a larger increase, relative to aged controls, in kidney TTR expression. As for the expression of other genes in monocytes, kidney TTR expression may be responsive to systemic signals resulting from developing amyloid pathology in the CNS. Liver showed little relative difference in expression with age in AD versus control mice.

Summary: MGAT3, TLR4, and VDR expression in monocytes, and kidney expression of TTR, may represent useful biomarkers of AD progression in mouse models, and effecting changes in expression of these genes may be useful for evaluating novel curcumin compounds tested for in vivo efficacy as potential AD drugs.

### Example 6

### AD Rodent Model Brain Slice System

A rodent brain slice model for AD has been developed where the expression of human APP by particle-mediated gene transfer (“biolistics”) leads to typical signs of neuronal dystrophy, and ultimately the degeneration and clearance of the cell body with a few days. Brain slice preparations offer a powerful experimental system because they preserve the three-dimensional tissue architecture and local environment of neurons and glia far better than dissociated cell culture. As such, drug lead candidates that show neuroprotective activity in brain-tissue based disease models have a much increased ability to predict efficacy in vivo and ultimately in the clinic.

Preparation of Brain Slice Explants. Cd Sprague-Dawley Rat Pups are Used at Postnatal day 10. Brains are cut into 250 μm coronal slices on a vibratome in chilled medium baths. Approximately 6 such slices can be cut from each rat brain; each are divided into “hemi-coronal” slices which are placed in individual 6-well transwell inserts. Slice cultures are maintained in transwell inserts suspended over serum-supplemented culture media at 32° C. in a humidified incubator under 5% CO₂.

Biolistic transfection. Particle-mediated gene transfer into brain slice explants is done using a commercial gene gun. 1.6 nm gold particles are used as the DNA carrier with a total DNA load of 4 μg DNA/mg Au, with the gene gun unit set at 95-105 psi at an aperture distance from the brain slices of ˜2.5 cm.

Plasmids and compounds. Full-length human APP (hAPP) was subcloned into an expression plasmid under the control of the CMV promoter. An expression construct for enhanced yellow fluorescent protein (EYFP) was made by transferring pEYFP-N1 into a similar expression vector. Slices transfected with EFYP only (maximal neuronal survival), or EYFP and hAPP (minimal neuronal survival), serve as controls.

Assessment of cortical neuron health. EYFP-expressing cortical neurons are identified based on their location within each brain slice explant and upon their characteristic morphology displaying a prominent apical dendrite, using a fluorescent stereomicroscope. Cortical neurons exhibiting normal-sized cell bodies, even and continuous expression of EYFP within all cell compartments, and a discernable apical dendrite >2 cell bodies long are scored as “healthy”. Representative images are captured using a digital camera on a fluorescence microscope. After 3 days, cortical neurons are scored. An experiment is considered successful if the difference of neuronal counts between EYFP only, and EYFP+hAPP, is ≧2.5 standard deviations (SD). Statistical significance of the neuronal counts in compound-treated slices is evaluated by ANOVA analysis, followed by post hoc Dunnett's analysis at the p≦0.05 confidence level.

Neuroprotective Effects of Curcumins in the Rat Brain Slice AD Model.

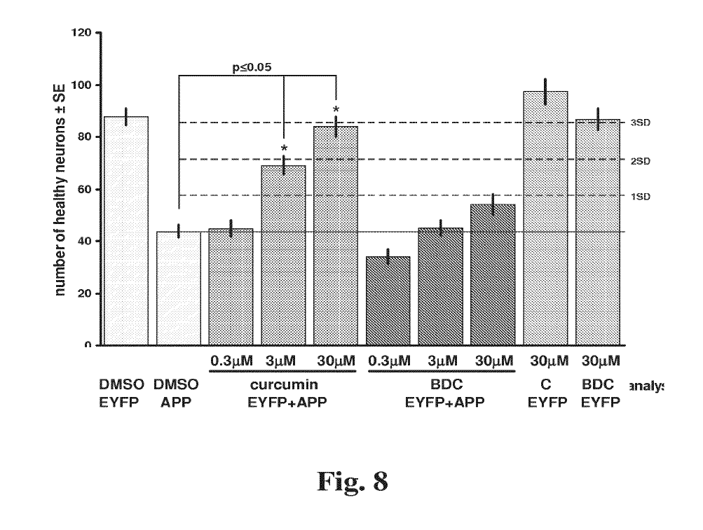

To date, we have tested purified natural Curcumin and BDC. For this purpose, rat brain slices were biolistically transfected with EFYP and hAPP and slices were cultured in medium containing three different concentrations of the respective compound. In two independent experiments with 12 brain slices per condition, Curcumin was able to prevent widespread neurodegeneration in a dose-dependent manner (FIG. 8, orange bars), that was statistically significant at the two highest concentrations (ANOVA followed by post-hoc Dunnett's comparisons at the p=0.05). BDC showed a dose-dependent trend for AD related neuroprotection, however this trend was not quite statistically significant, indicating that slightly higher concentrations were more suitable for testing (FIG. 8, blue bars). Importantly, the neuroprotective effects of the curcumins were specific for hAPP-induced neurodegeneration, as neither compound showed any effects on control brain slices. These results illustrate the value of this functional ex vivo assay for evaluating natural curcumins and derived synthetic analogs. before in vivo work.

### Example 7

### Gene Expression Biomarkers of Oxidative Stress, and Protective Effects of Curcumins

The pathogenesis of AD is thought to be linked to both Aβ deposition and oxidative stress, though it is unclear how these factors lead to neurodegeneration. It has been reported that Aβ may induce membrane-associated oxidative stress, and the resulting altered lipid metabolism and disruption of cellular calcium homeostasis may trigger apoptosis in various neurodegenerative disorders. ALS pathogenesis is also believed to involve defects in the cellular response to oxidative stress, as the gene encoding superoxide dismutase (SOD1), a major cytoplasmic antioxidant enzyme, is frequently mutated in familial forms of ALS. Identifying common mechanisms defective in different neurodegenerative disorders would significantly advance therapeutic development. Natural curcumin has been reported to have anti-oxidant activity. We have therefore evaluated the in vitro expression of MGAT3, TLR, and VDR genes as potential markers of response to oxidative stress in various cultured cells, including cells derived from ALS patients and controls. We also tested curcumin (BDC) for protective effects against oxidative stress.

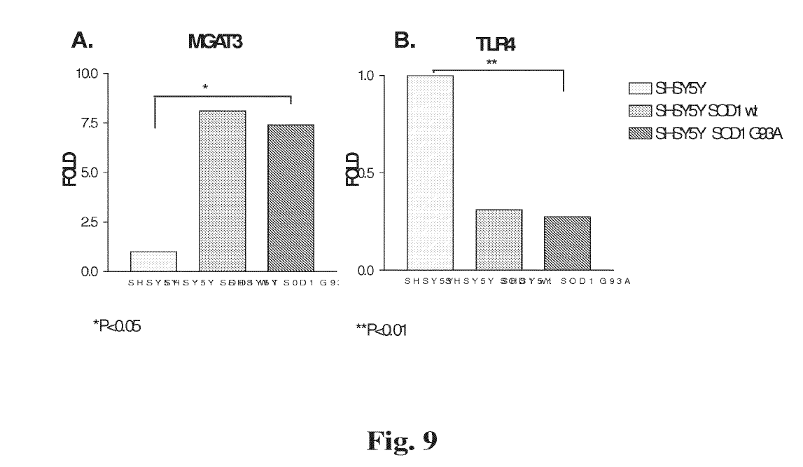

Expression in neuroblastoma cells in relation to SOD1 expression. Baseline expression of these genes was tested by qPCR (as in Example 2) in a neuroblastoma cell line (SHSY5Y), and in derivative lines that overexpress a transfected wild type (wt) or a transfected mutant (G93A) SOD1 gene (i.e. “SOD⁺”). Representative results are shown in FIG. 9, in which significantly increased expression of MGAT3 was seen in both cell lines overexpressing wt or mutant SOD1, compared to untransfected SHSY5Y cells. Other tested genes showed either similar expression increases correlating with SOD⁺ (TLR2, TLR3), decreased expression in SOD⁺ lines relative to untransfected cells (TLR4, FIG. 9B), or little or no change in relation to SOD1 expression (VDR). The results of gene expression in these cell lines are summarized in Table 3.

TABLE 3
Relative gene expression changes in neuroblastoma
cells with normal and overexpressed SOD1.

| | Compound | | |
|---|---|---|---|
| | | SH- | SH- |
| Gene | SHSY5Y | wtSOD⁺ | (G93A)SOD⁺ |
| MGAT3 | 1 | ++ | ++ |
| TLR2 | 1 | ++ | + |
| TLR3 | 1 | + | − |
| TLR4 | 1 | − | − |
| VDR | 1 | − | 1 |

mRNA levels determined by qPCR in SOD⁺ lines are shown relative control SHSY5Y cells (“1”).
‘+’, increased relative expression; ‘−’, decreased expression.

The data support relationships between expression of the different tested genes and the antioxidant enzyme SOD1, suggesting possible involvement of certain tested genes in common cellular responses to oxidative stress. SOD1 expression appears to potentiate expression of MGAT3 and at least two TLR genes, whereas TLR4 and VDR did not show corresponding increases and thus may not participate directly in this response.

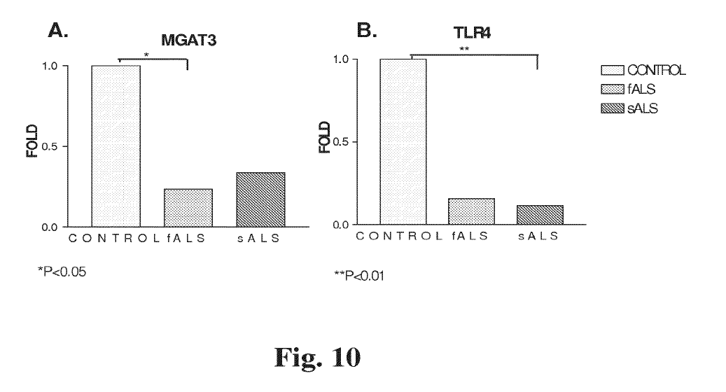

Gene expression in ALS samples. We also examined expression of these genes in fibroblast cell lines derived from eight patients representing either familial or sporadic forms of ALS, and from two control individuals. The ALS fibroblast lines were derived from patients carrying either wt or mutant SOD1 genes. FIG. 10 shows representative qPCR results for all tested genes, indicating significantly reduced expression of MGAT3 and TLR4 in all ALS relative to control fibroblast lines, regardless of familial versus sporadic or SOD1 status. Similar results were obtained for TLRs 2 and 3, and VDR. These results reveal an association between altered expression of these genes and ALS patient samples. Thus expression profiles of these genes in samples easily obtained through skin biopsy may be useful as markers of ALS susceptibility or onset. Affecting expression changes of these genes in fibroblasts or other tissues may be useful for evaluating novel compounds as potential drugs for treating ALS, AD, or other neurodegenerative diseases or disorders involving defects in response to oxidative stress.

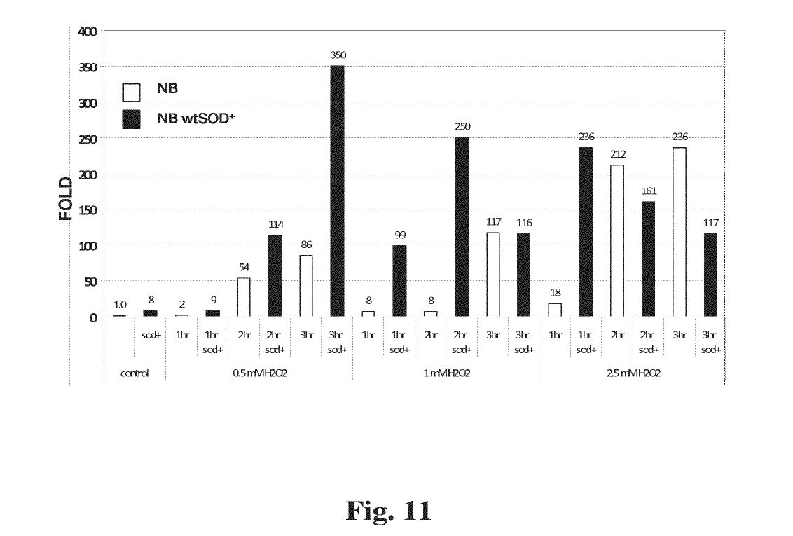

Gene expression in response to oxidative stress. Having established associations between expression of these genes and both SOD1 expression and ALS patient samples, we directly determined in vitro gene expression in response to experimentally induced oxidative stress. The neuroblastoma SHSY5Y and wtSOD⁺ overexpressing cells were subjected to varying concentrations of hydrogen peroxide (H₂O₂; 0.5-5 mM) for varying times. After 1, 2, and 3 hours of incubation, cells were harvested, total RNAs were isolated, and prepared cDNAs were used in qPCR experiments to quantify normalized mRNA levels. Results for MGAT3 expression in these cells in response to H₂O₂ is shown in FIG. 11. MGAT3 expression in untransfected cells increased in relation to oxidative stress conditions, with peak expression (>200-fold increased) at 2-3 hours in the presence of 2.5 mM H₂O₂. Again, increased SOD1 expression appeared to potentiate MGAT3 expression, with higher maximal MGAT3 expression (>300-fold increased) observed in the wtSOD⁺ line, and at lower H₂O₂ concentrations (e.g. 0.5 mM). In a similar experiment, it was observed that curcumin (BDC) can enhance MGAT3 expression in response to oxidative stress (not shown). These results showed that MGAT3 expression may be a useful biomarker of the cellular response(s) to oxidative stress, and may be used as a marker for evaluating the effects of potential AD or ALS drugs upon these responses and upon cellular viability. Further, curcumins may potentiate the cellular response to oxidative stress and may therefore have a protective effect.

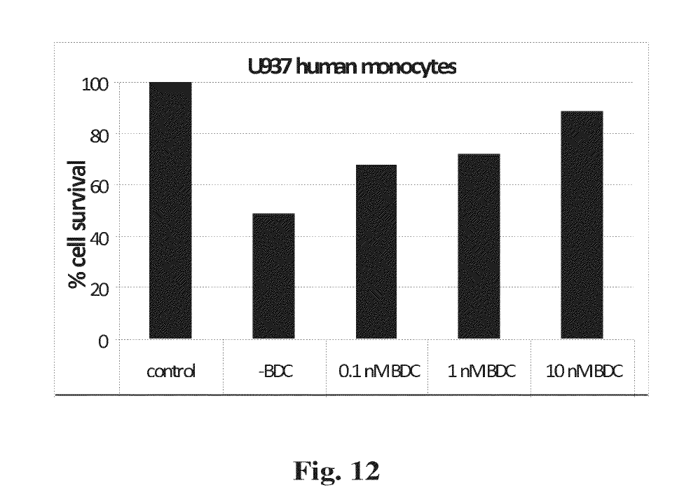

Protective effect of curcumin against oxidative stress. We tested the potential of curcumin to protect cells in vitro under conditions of oxidative stress. Natural curcumin has been reported to have antioxidant activity and to scavenge free radicals, but reports of the direct effects of curcumin or analogs upon cell viability under conditions of oxidative stress are limited. Viability of the human monocytic cell line U-937 was evaluated in the presence of H₂O₂, plus varying concentrations of natural BDC (range 0-10 nM). We observed that these cells are more sensitive to oxidative stress than are the neuroblastoma SHSY5Y cells, and so reduced concentrations of H₂O₂ were used for this experiment. Using Trypan-blue staining to quantify cell viability, a concentration of only 100 μM H₂O₂ resulted in approximately 50% cell death after two hours (FIG. 12). However, concentrations of BDC between 0.1-10 nM (i.e. similar concentration range inducing maximal gene expression increases +Aβ in these cells) increased 2-hour cell survival rates up to 90%. Cell viability decreased above this concentration, suggesting a toxic effect by BDC in combination with H₂O₂. These results indicate the potential utility of BDC and other curcumins, not only for binding reactive free radicals, but for protection of living cells under conditions of oxidative stress, as has been described for neurons exposed to Aβ in AD, and possibly in other neurodegenerative diseases. Assays such as Trypan-blue staining, or other biochemical measures of cell toxicity/survival (e.g. ATP levels) may be useful for evaluating curcumin analogs for their neuroprotective effects against oxidative stress and as candidate AD or ALS drugs.

### Example 8

### Synthesis of Novel Immunomodulating Compounds

Curcumin analogs. Bisdesmethoxycurcuminoid is among the most potent immunoenhancing curcuminoid compounds identified, which also up-regulates MGAT3 and TLR transcription (Proc Natl Acad Sci USA 104 (2007) 12849). General Synthesis. Compounds described in the present invention can be prepared following schemes 1-3 by a person skilled in the art.

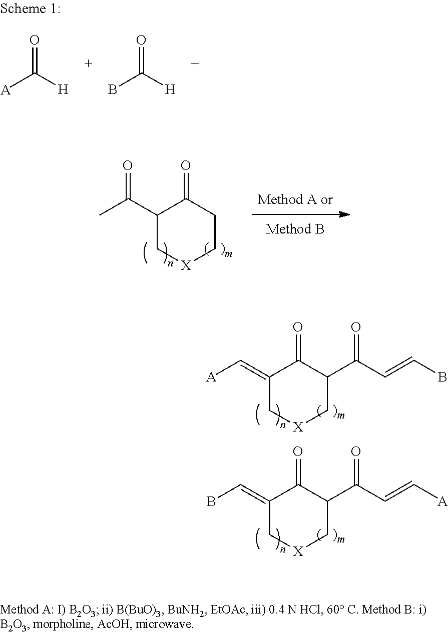

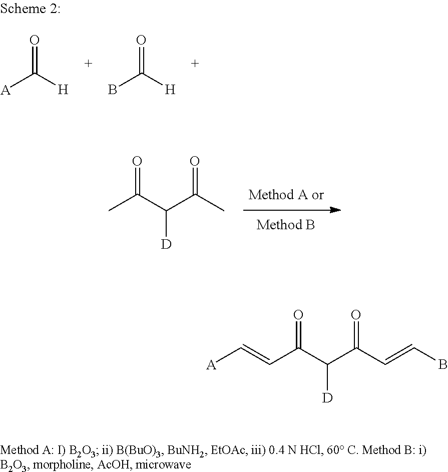

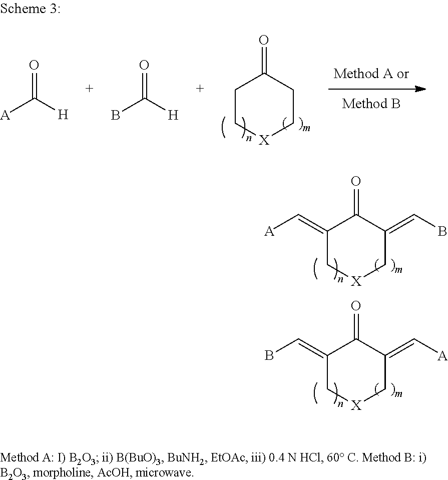

Method A (Eur J. Med Chem, 1997, 32, 321-328): A mixture of acetylacetone, acetylcyclohexanone, or cyclic ketone (19.5 mmol) and boric anhydride (12.8 mmol) was stirred at room temperature for 1 h under an atmosphere of argon. The aldehyde (40 mmol) and tributyl borate (80 mmol) were dissolved in dry ethyl acetate (50 mL) in a round bottom flask under an atmosphere of argon. The boron complex was added to this mixture and the reaction mixture was stirred at room temperature for 5-10 min n-Butylamine (0.4 mL) was added in 0.1 mL portions every 10 minutes. The resulting mixture was stirred for another 4 h and then allowed to stand overnight. The reaction was heated to 60° C. and hydrochloric acid (0.4 N, 30 ml) was added. After stirring at 60° C. for 1 h, the layers were separated and the organic layer was successively washed with water and brine. The solution was dried over Na₂SO₄, filtered and concentrated to dryness. Crude product was purified by column chromatography, preparative TLC, or by precipitation.

Method B (ARKIVOC 2006, (xiii), 64-72): To a mixture of acetylacetone, acetylcyclohexanone, or cyclic ketone (0.13 mL, 1.0 mmol) and boric anhydride (70 mg, 1.0 mmol) in a pressure tube was added 4-dimethylaminobenzaldehyde (300 mg, 2.0 mmol), morpholine (0.01 mL) and acetic acid (0.01 mL). The resulting mixture was heated in a conventional microwave at highest power for 1 min. The precipitate was filtered and washed with cold methanol. The precipitate was further purified using a short column (hexane/EtOAc, 1:1, v/v) to remove baseline impurity. A and B substitutents can be further modified to obtain the desired properties such as activity and solubility.

Subsequent to the identification of bisdemethoxycurcumin (5-Hydroxy-1,7-bis-(4-hydroxy-phenyl)-hepta-1,4,6-trien-3-one) as the most active fraction in the bioassay-guided fractionation, it was independently synthesized and tested. It too showed considerable activity.

5-Hydroxy-1,7-bis-(4-hydroxy-phenyl)-hepta-1,4,6-trien-3-one (Bisdemethoxycurcumin), (1). Bisdemethoxycurcumin was prepared according to Method A using the appropriate ketone and aldehyde. The crude product was dissolved in ethylacetate (75 mL) and methanol (50 mL) and the solution was stored at −20° C. overnight to precipitate the product. The precipitate was washed with cold methanol.

1,7-Bis(4-hydroxy-3-methoxyphenyl)-5-hydroxy-hepta-1,4,6-trien-3-one (curcumin), (2). Compound 2 was prepared according to Method A from the appropriate ketone and aldehyde The crude product was purified by flash column chromatography (hexane/EtOAc 2:1).

1,7-Bis(4-dimethylamino-2-methoxyphenyl)-5-hydroxy-hepta-1,4,6-trien-3-one, (3). Compound 3 was prepared according to Method A from the appropriate ketone and aldehyde. The crude product was purified by flash column chromatography (hexane/EtOAc 2:1.

4-Ethyl-5-hydroxy-1,7-bis(4-hydroxyphenyl)hepta-1,4,6-trien-3-one, (4).

Compound 4 was prepared according to Method A from the appropriate ketone and aldehyde. The crude product was recrystallized from ethyl acetate and methanol (3:2). The crystals were washed with cold ethyl acetate.

5-Hydroxy-1,7-bis(4-hydroxyphenyl)hept-4-en-3-one, (5).

A mixture of bisdemethoxycurcumin 1 (100 mg, 0.32 mmol) and Pd/C (19 mg) was suspended in ethyl acetate (15 mL). The mixture was placed under an atmosphere of hydrogen, and the mixture was stirred at room temperature overnight. The black precipitate was filtered off and the colorless solution was concentrated to dryness.

2-(4-Hydroxybenzylidene)-6-(3-(4-hydroxyphenyl)acryloyl)cyclohexanone, (6).

Compound 6 was prepared according to Method A from the appropriate ketone and aldehyde. The crude product was purified by flash chromatography (hexane/EtOAc, 2:1) followed by recrystallization from EtOAc and MeOH.

2-(4-(Dimethylamino-2-methoxybenzylidene)-3-(4-(dimethylamino)-2-methoxyphenyl)-1-hydroxyallylidene)cyclohexanone, (7).

Compound 7 was prepared according to Method A from the appropriate ketone and aldehyde.

The crude product was purified by flash chromatography (hexane/EtOAc 3:1).

2-(3-Cyclohexylacryloyl)-6-(cyclohexylmethylene)cyclohexanone, (8).

Compound 8 was prepared according to Method A from the appropriate ketone and aldehyde.

The crude product was purified by flash column chromatography (hexane/ether 20:1).

1,7-bis(4-Dimethylaminophenyl)-4-ethyl-hepta-1,6-diene-3,5-dione, (9).

Compound 9 was prepared according to Method B from the appropriate ketone and aldehyde. After the reaction mixture was cooled to room temperature, methanol was added and the mixture was sonicated. The precipitate was filtered and washed with cold methanol. The precipitate was further purified using a short column (hexane/EtOAc 1:1) to remove baseline impurity.

2-(4-(Dimethylamino)benzylidene)-3-(4-(dimethylamino)phenyl)-1-hydroxyallylidene)cyclohexanone, (10). Compound 10 was prepared according to Method B from the appropriate ketone and aldehyde. The precipitate was purified using a short flash column chromatography (hexane/EtOAc 2:1).

2-(5-Fluoro-2-methoxybenzylidene)-3-(5-fluoro-2-methoxypenyl)-1-hydroxyallylidene)cyclohexanone, (11). Compound 11 was prepared according to Method B from the appropriate ketone and aldehyde.

4-Ethyl-1,7-bis(5-fluoro-2-methoxyphenyl)-hepta-1,6-diene-3,5-dione, (12).

Compound 12 was prepared according to Method B from the appropriate ketone and aldehyde.

1,7-Bis(4-(dimethylamino)-2-methoxyphenyl)-4-ethyl-hepta-1,6-diene-3,5-dione, (13). Compound 13 was prepared according to Method B from the appropriate ketone and aldehyde. The crude product was purified using flash chromatography.

1,7-bis(4-(Trifluoromethyl)phenyl)-5-hydroxyhepta-1,4,6-trien-3-one, (14).

Compound 14 was prepared according to Method B from the appropriate ketone and aldehyde. The crude product was purified using flash chromatography (EtOAc/hexane 12% then 20%).

3,5-bis(4-Hydroxybenzylidene)dihydro-2H-pyran-4(3H)-one, (15).

Compound 15 was prepared according to Method B from the appropriate ketone and aldehyde. The crude was purified by flash chromatography (hexane/EtOAc 1:1).

5-Hydroxy-1,7-bis-(4-methoxy-phenyl)-hepta-1,4,6-trien-3-one, (16).

Compound 16 was prepared according to Method A from the appropriate ketone and aldehyde.

Acetic acid 4-[7-(4-acetoxy-phenyl)-5-hydroxy-3-oxo-hepta-1,4,6-trienyl]-phenyl ester, (17). Compound 1 was treated with a mixture of acetyl chloride/triethylamine (4:2) to give compound 17.

2,2-Dimethyl-propionic acid 4-{7-[4-(2,2-dimethyl-propionyloxy)-phenyl]-3,5-dioxo-hepta-1,6-dienyl}-phenyl ester, (18). Compound 1 was treated with a mixture of pivaloyl chloride/triethylamine (4:2) to give compound 18 as a yellow powder.

5-Hydroxy-1,7-bis-(3-hydroxyphenyl)-hepta-1,4,6-trien-3-one, (19).

Compound 19 was prepared according to Method A from the appropriate ketone and aldehyde.

1,7-bis-(4-Dimethylaminophenyl)-5-hydroxy-hepta-1,4,6-trien-3-one, (20).

Compound 20 was prepared according to Method A from the appropriate ketone and aldehyde.

5-Hydroxy-1,7-bis-(3-hydroxy-4-methoxyphenyl)-hepta-1,4,6-trien-3-one, (21).

Compound 21 was prepared according to Method A from the appropriate ketone and aldehyde.

5-Hydroxy-1,7-bis-(4-hydroxy-2-methoxyphenyl)-hepta-1,4,6-trien-3-one, (22).

Compound 22 was prepared according to Method A from the appropriate ketone and aldehyde.

5-Hydroxy-1,7-bis-(2-hydroxy-4-methoxyphenyl)-hepta-1,4,6-trien-3-one, (23).

Compound 23 was prepared according to Method A from the appropriate ketone and aldehyde.

1,7-bis-(3-Chloro-4-hydroxyphenyl)-5-Hydroxy-hepta-1,4,6-trien-3-one, (24).

Compound 24 was prepared according to Method A from the appropriate ketone and aldehyde.

5-Hydroxy-1,7-bis-(2-methoxyphenyl)-hepta-1,4,6-trien-3-one, (25).

Compound 25 was prepared according to Method A from the appropriate ketone and aldehyde.

1,7-bis-(5-Fluoro-2-methoxyphenyl)-5-hydroxy-hepta-1,4,6-trien-3-one, (26).

Compound 26 was prepared according to Method A from the appropriate ketone and aldehyde.

5-Hydroxy-1,7-diphenylhepta-1,4,6-trien-3-one, (27). Compound 27 was prepared according to Method A from the appropriate ketone and aldehyde.

5-Hydroxy-1,7-ditolylhepta-1,4,6-trien-3-one, (28).

Compound 28 was prepared according to Method A from the appropriate ketone and aldehyde.

1,7-bis(4-Methylthiophenyl)-5-hydroxyhepta-1,4,6-trien-3-one, (29).

Compound 29 was prepared according to Method A from the appropriate ketone and aldehyde.

1,7-bis(4-tert-Butylphenyl)-5-hydroxyhepta-1,4,6-trien-3-one, (30).

Compound 30 was prepared according to Method A from the appropriate ketone and aldehyde.

1-(4-(Dimethylaminophenyl)-7-(5-fluoro-2-methoxyphenyl)hepta-1,6-diene-3,5-dione, (31). Acetylacetone (2 mL, 20 mmol) was dissolved in ethyl acetate (4 mL). Boric anhydride (0.97 g, 14 mmol) was added and the mixture was stirred at 70° C. for 40 min 4-Dimethylaminobenzaldehyde (0.99 g, 6.7 mmol) and tributyl borate (1.8 mL, 6.7 mmol) was added to this solution. The resulting mixture was stirred at 70° C. for 30 min Butylamine (0.66 mL, 6.7 mmol) was added dropwise over a period of 20 min and the solution was stirred at 100° C. for 1 h. To this mixture was added 1N HCl and the solution was stirred at 50° C. for 30 min. The solution was extracted with EtOAc and the resulting crude product was purified by chromatography (hexane/EtOAc, 2:1, v/v) to give 1-(4-(dimethylamino)phenyl)-5-hydroxyhexa-1,4-dien-3-one as a yellow solid. This intermediate (100 mg, 0.43 mmol) and boric anhydride (21 mg, 0.3 mmol) were dissolved in ethylacetate (4 mL) and stirred at 70° C. for 30 min. To this solution were added 5-fluoro-2-methoxybenzaldehyde (64.5 mg, 0.43 mmol) and tributylborate (0.23 mL, 0.86 mmol). The resulting mixture was stirred for another 30 min at 70° C. Piperidine (0.04 mL, 0.43 mmol) was added dropwise and the mixture was stirred at 100° C. for 1 h then cooled to 60° C. and 1N HCl was added to reach pH 3-4. The solution was stirred at 60° C. for 40 min and then extracted with ethyl acetate. The crude product was purified by preparative TLC (hexane/EtOAc, 2:1, v/v) to give the compound 31 as yellow solid.

2-(1-Hydroxy-3-(4-hydroxyphenyl)allylidene)-5-(4-hydroxybenzylidene) cyclopentanone, (32). Compound 32 was prepared according to Method B from the appropriate ketone and aldehyde.

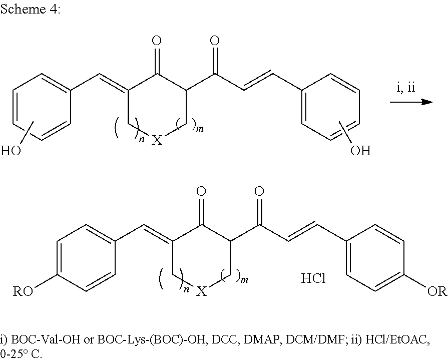

4,4′-(3,5-Dioxohepta-1,6-diene-1,7-diyl)bis(4,1-phenylene) bis(2-amino-3-methylbutanoate) HCl salt, (33).

Following Scheme 4, a mixture of bisdemethoxycurcumin 1 (50 mg, 0.16 mmol), dicyclohexylcarbodiimide (84 mg, 0.4 mmol), dimethylaminopyridine (4 mg, 0.03 mmol) and BOC-Val-OH (141 mg, 0.65 mmol) were dissolved in dichloromethane (13 mL) and DMF (0.5 mL) in a dry flask and stirred at room temperature under Ar(g) for 2 h. The white precipitate was filtered off and solvent was removed in vacuo. The oily residue was redissolved in dichloromethane and water. The solution was extracted with dichloromethane; organic layers were dried over Na₂SO₄. The solution was filtered and concentrated. The resulting oil was purified using preparative TLC (hexane/EtOAc 3:1) to give 60 mg (53%) of the diester. The BOC-protected diester, (56 mg, 0.08 mmol) was dissolved in EtOAc (4 mL) and dichloromethane (0.4 mL). The yellow solution was cooled to 0° C. in an ice bath. HCl(g) was bubbled through the solution for 2 minutes. The vial was capped and solution was stirred for 3 h. Solvent was removed under stream of argon, and the residue was washed with ether to give the 42 mg (91%) hydrochloride salt.

4,4′-(3,5-Dioxohepta-1,6-diene-1,7-diyl)bis(4,1-phenylene) bis(2,6-diaminohexanoate) HCl salt, (34). Compound 34 was synthesized according the procedure described for compound 33 using Boc-Lys(Boc)-OH in place of Boc-Val-OH.

4,4′-(3,5-Dioxohepta-1,6-diene-1,7-diyl)bis(2-methoxy-4,1-phenylene)bis(2-amino-3-methylbutanoate) HCl salt, (35). Compound 35 was synthesized according the procedure described for compound 33 beginning with curcumin

4,4′-(3,5-dioxohepta-1,6-diene-1,7-diyl)bis(2-methoxy-4,1-phenylene)bis(2,6-diaminohexanoate) HCl salt, (36). Compound 36 was synthesized according the procedure described for compound 34 beginning with curcumin to give quantitative yield of compound 36 as a tetrahydrochloride salt.

4-(4-(2-Amino-3-methylbatanoyloxy)benzylidene)-2-oxocyclohexyl)-3-oxoprop-1-enyl)phenyl-2-amino-3-methylbatanoate HCl salt, (37). Compound 37 was prepared from compound 6 following the procedure used to prepare compound 33.

4-(3-(4-(2,6-Diaminohexanoyloxy)benzylidene)-2-oxocyclohexyl)-3-oxoprop-1-enyl)phenyl-2,6-diaminohexanoate, HCl salt, (38). Compound 38 was synthesized according to the procedure described for compound 34 beginning with compound 6 to give the product as the tetrahydrochloride salt.

2-(4-Dimethylamino-3-hydroxy-benzylidene)-6-[3-(4-dimethylamino-3-hydroxy-phenyl)-1-hydroxy-allylidene]-cyclohexanone, (39). Compound 39 was prepared according to Method A using the appropriate ketone and aldehyde. Crude product was purified by preparative TLC (hexane/EtOAc 1:1).

5-(3-(3-(3-(2-Amino-3-methylbatanoyloxy)-4-(dimethylamino)benzylidene)-2-oxocyclohexyl)-3-oxoprop-1-enyl)-2-(dimethylamino)phenyl 2-amino-3-methylbatanoate, HCl salt, (40). Compound 40 was prepared from compound 39 according to the preparation of compound 33. The resulting solid was dried under high vacuum.

2-(4-(Dimethylamino)benzylidene)-6-(3-(4-hydroxyphenyl)acryloyl)cyclohexanone, (41). Compound 41 was prepared according to Method B using the appropriate ketone and aldehydes. Specifically, 2-(3-(4-hydroxyphenyl)acryloyl)cyclohexanone, (50 mg, 0.2 mmol) and boric anhydride (14.3 mg, 0.2 mmol) were mixed in 50 mL Erlenmeyer flask. 4-Dimethylaminobenzaldehyde (30 mg, 0.2 mmol), morpholine (0.1 mL) and acetic acid (0.1 mL) were added successively. The resulting mixture was heated in a microwave at the highest power for 1 min. Methanol was added and the mixture was sonicated. Methanol was removed and the resulting crude product was purified by flash chromatography (hexane/EtOAc 1:1) to give 18 mg (24%) of a yellow solid.

4-(3-(3-(4-(Dimethylamino)benzylidene)-2-oxocyclohexyl)-3-oxoprop-1-enyl)phenyl 2-amino-3-methylbatanoate, HCl salt, (42). Compound 42 was prepared from compound 41 according to the preparation of compound 33.

2-(4-Hydroxy-3-methoxybenzylidene)-6-(3-(4-hydroxy-3-methoxyphenyl)acryloyl)cyclohexanone, (43). Compound 43 was prepared according to Method B using the appropriate ketone and aldehyde. The product was purified by flash chromatography (hexane/EtOAc 3:2).

4-(3-(3-(4-(2-Amino-3-methylbatanoyloxy)-3-methoxybenzylidene)-2-oxocyclohexyl)-3-oxoprop-1-enyl)-2-methoxyphenyl 2-amino-3-methylbutanoate, (44). Compound 44 was prepared from compound 43 following the procedure to make compound 33.

4-(3-(3-(4-(2,6-Diaminohexanoyloxy)-3-methoxybenzylidene)-2-oxocyclohexyl)-3-oxoprop-1-enyl)-2-methoxyphenyl 2,6-diaminohexanoate, (45). Compound 45 was prepared from compound 43 following the procedure to make compound 34.

2-(4-Pyrrolidin-1-ylbenzylidene)-6-(3-(4-pyrrolidin-1-ylphenyl)acryloyl)cyclohexanone, (46). Compound 46 was prepared according to Method B from the appropriate ketone and aldehyde. The product was precipitated from methanol at −20° C. Precipitate was isolated by filtration and washed with cold methanol.

2-(4-Pyrazol-1-ylbenzylidene)-6-(3-(4-pyrazol-1-ylphenyl)acryloyl)cyclohexanone, (47). Compound 47 was prepared according to Method B from the appropriate ketone and aldehyde. The product was precipitated from methanol at −20° C. Precipitate was isolated by filtration and washed with cold methanol.

(2-(4-Morpholinobenzylidene)-6-(3-(4-morpholinophenyl)acryloyl)cyclohexanone, (48).

Compound 48 was prepared according to Method B from the appropriate ketone and aldehyde. Crude product was purified by column chromatography on silica gel (hexane/EtOAc 1:1). Major fractions were collected and purified again by preparative TLC (hexane/EtOAc 3:2).

(2-(4-(Piperidin-1-yl)benzylidene)-6-(3-(4-(piperidin-1-yl)phenyl)acryloyl)cyclohexanone, (49). Compound 49 was prepared according to Method B from the appropriate ketone and aldehyde.

Following the same procedures, the following compounds were made:
6-hydroxyhepta-2,4-dienoyl)-6-(4-hydroxypent-2-enylidene)cyclohexanone;
(3-methylbutylidene)-5-methylhex-2-enoyl)cyclohexanone;
3-(4-hydroxycyclohexyl)acryloyl)-6-((4-hydroxycyclohexyl)methylene)cyclohexanone;
1-(4-hydroxybenzyl)-N-isopentylpiperidine-3-carboxamide;
N-(3-hydroxy-3-methylbutyl)-1-(4-hydroxybenzyl)piperidine-3-carboxamide;
4,4′-3-hydroxy-5-oxohepta-1,3,6-triene-1,7-diyl)bis(4,1-phenylene)bis(1-aminocyclo propanecarboxylate);
4-(3-(4-(2-amino-3-methylbutanoyloxy)-3-methoxybenzylidene)-2-oxocyclohexylidene)-3-hydroxyprop-1-enyl)-2-methoxyphenyl 2-amino-3-methylbutanoate;
4-(3-(4-(2,6-diaminohexanoyloxy)-3-methoxybenzylidene)-2-oxocyclohexylidene)-3-hydroxyprop-1-enyl)-2-methoxyphenyl 2,6-diaminohexanoate;
2-(4-(dimethylamino)benzylidene)-6-(3-(4-hydroxyphenyl)acryloyl)cyclohexanone;
2-(4-(dimethylamino)-3-hydroxybenzylidene)-6-(3-(4-(dimethylamino)-3-hydroxyphenyl)acryloyl)cyclohexanone;
4-(3-(3-(4-(dimethylamino)benzylidene)-2-oxocyclohexyl)-3-oxoprop-1-enyl)phenyl-2-amino-3-methylbutanoate;
4-(3-(3-(4-(dimethylamino)benzylidene)-2-oxocyclohexyl)-3-oxoprop-1-enyl)phenyl-2,6-diaminohexanoate;
5-(3-(3-(3-(2-amino-3-methylbutanoyloxy)-4-(dimethylamino)benzylidene)-2-oxocyclohexyl)-3-oxoprop-1-enyl)-2-(dimethylamino)phenyl 2-amino-3-methylbutanoate;
5-(3-(3-(3-(2,6-diaminohexanoyloxy)-4-(dimethylamino)benzylidene)-2-oxocyclohexyl)-3-oxoprop-1-enyl)-2-(dimethylamino)phenyl 2,6-diaminohexanoate.

Structures and Compositional Analysis of Synthetic Curcuminoids.

Tables 4 and 5 show chemical structures and data from ESI-mass spec (m/z) and ¹H-NMR analysis of synthesized compounds.

TABLE 4
Structures of synthesized curcumin analogs.

| Cpd | |
|---|---|
| # | Structure |
| 1 | 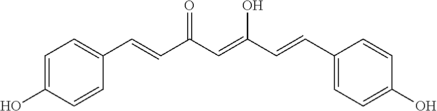 |
| 2 | 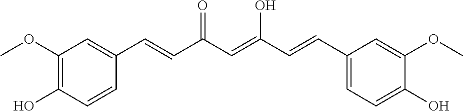 |
| 3 | 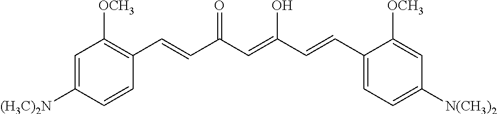 |
| 4 | 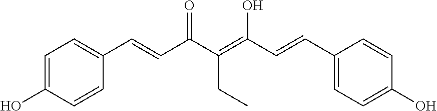 |
| 5 | 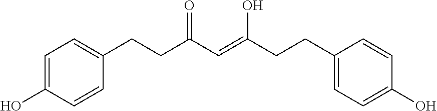 |
| 6 | 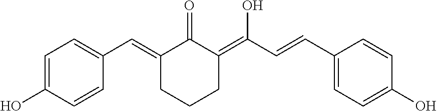 |
| 7 | 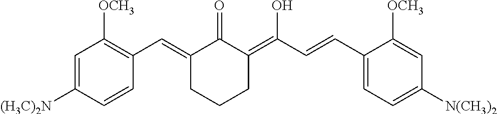 |
| 8 | 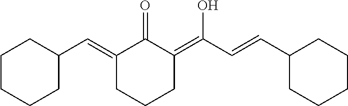 |
| 9 | 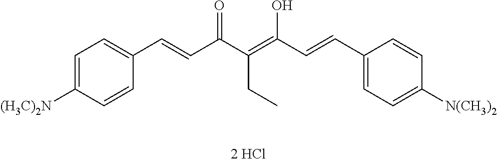 |
| 10 | 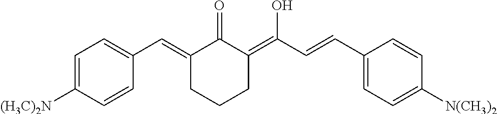 |
| 11 | 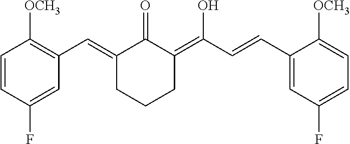 |
| 12 | 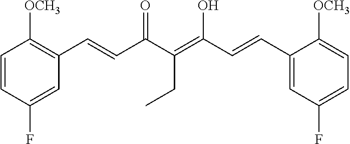 |
| 13 | 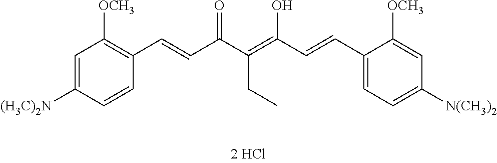 |
| 14 | 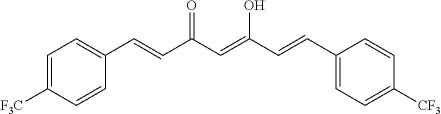 |
| 15 | 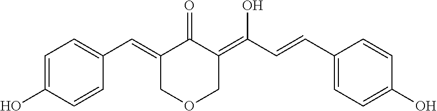 |
| 16 | 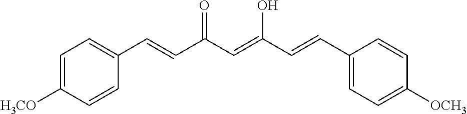 |
| 17 | 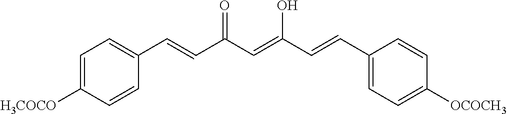 |
| 18 | 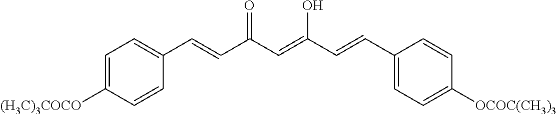 |
| 19 | 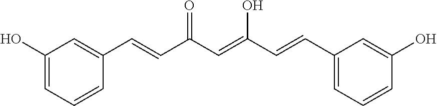 |
| 20 | 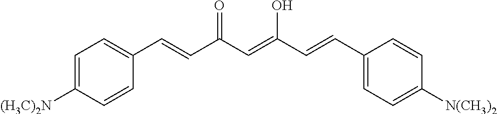 |
| 21 | 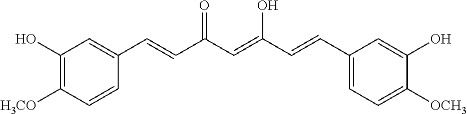 |
| 22 | 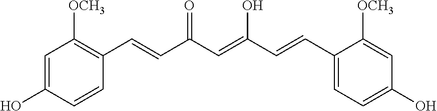 |
| 23 | 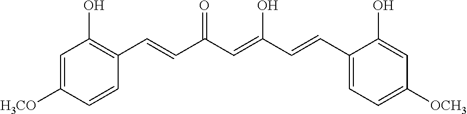 |
| 24 | 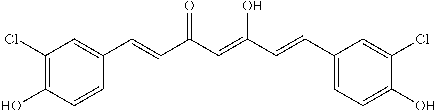 |
| 25 | 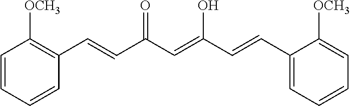 |
| 26 | 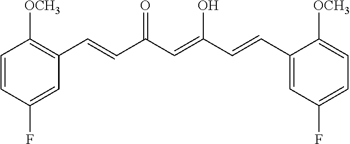 |
| 27 | 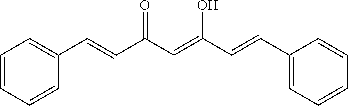 |
| 28 | 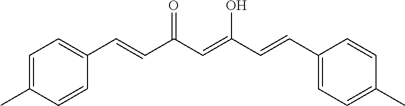 |
| 29 | 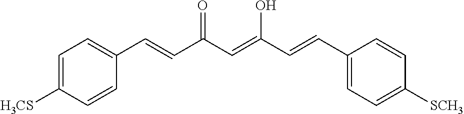 |
| 30 | 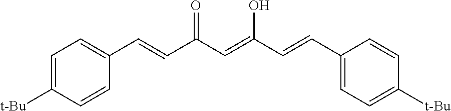 |
| 31 | 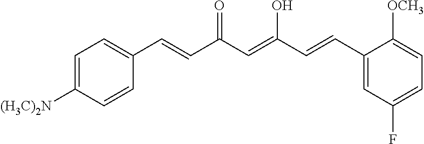 |
| 32 | 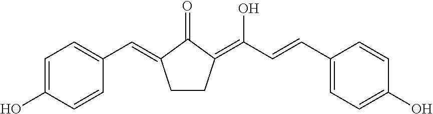 |
| 33 | 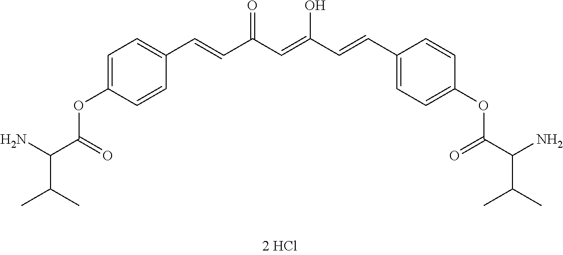 |
| 34 | 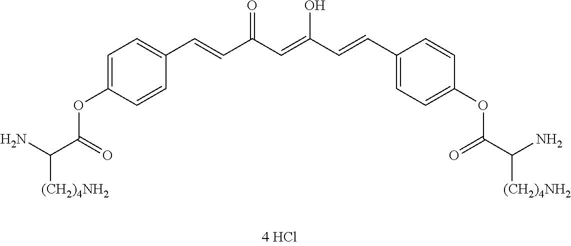 |
| 35 | 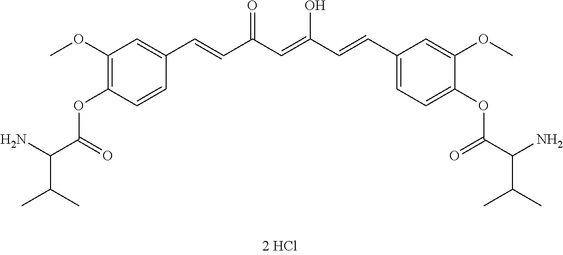 |
| 36 | 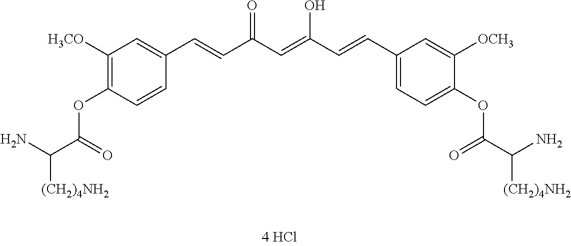 |
| 37 | 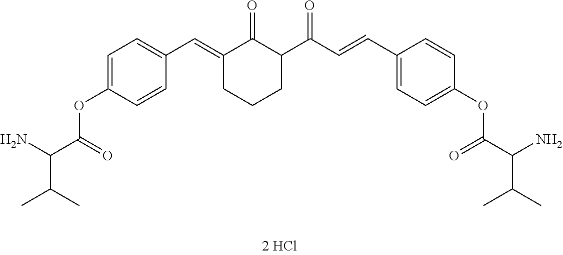 |
| 38 | 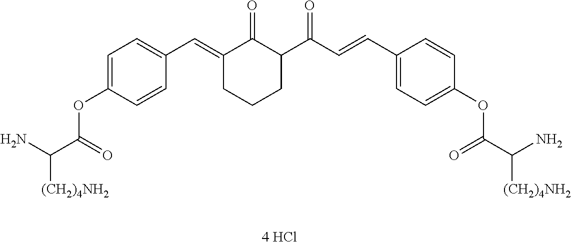 |
| 39 | 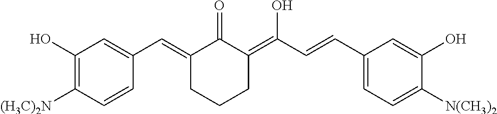 |
| 40 | 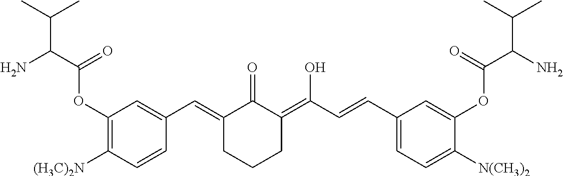 |
| 41 | 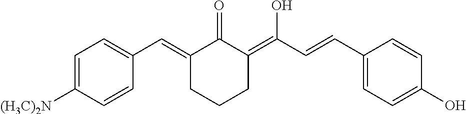 |
| 42 | 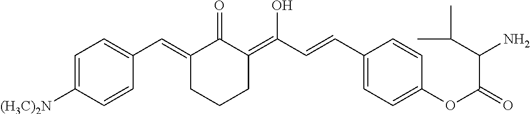 |
| 43 | 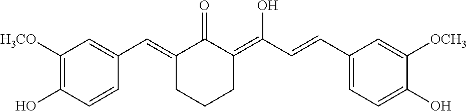 |
| 44 | 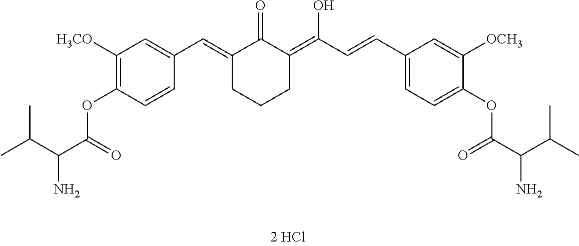 |
| 45 | 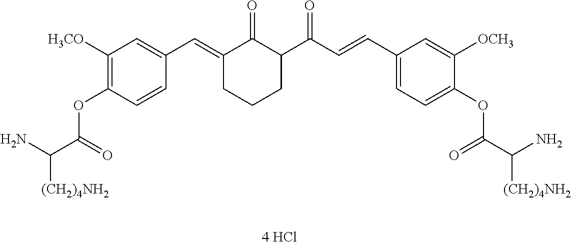 |
| 46 | 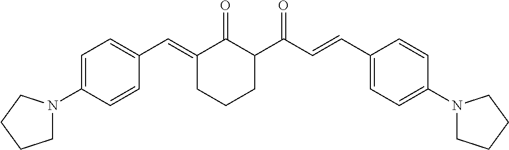 |
| 47 | 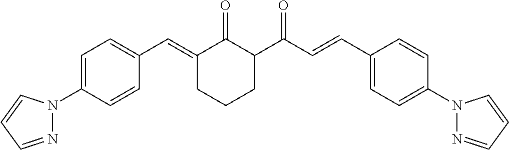 |
| 48 | 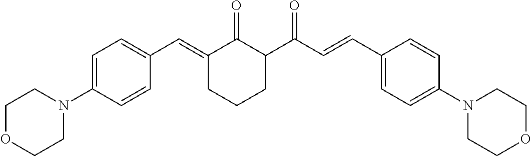 |
| 49 | 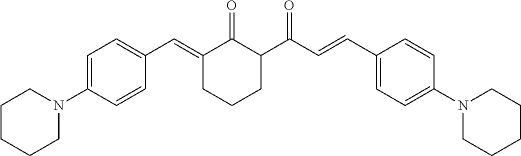 |

TABLE 5
Analysis of synthesized curcuminoids by mass spectrometry and NMR.

| Cpd # | ESI-MS m/z | ¹H NMR ^{a} |
|---|---|---|
| 1 | 309 (MH+), 331 (MNa+), 307 | δ 7.50 (d, 2H, Ph—CH—), 7.29 (m, 4H, Ph), 6.62-6.78 (m, 4H, |
| | (MH)− | Ph), 6.37 (d, 2H, —CH—), 5.70 (s, 1H, —CO—CH—CO—) |
| 2 | 369 (MH⁺), 367 (M⁻) | δ 7.41 (d, J = 15.6 Hz, 2H, Ph—CH—), 6.92 (m, 4H, Ph), 6.72 |
| | | (m, 2H, Ph), 6.34 (d, J = 15.6 Hz, 2H, —CH—), 5.69 (s, 1H, |
| | | —CO—CH—CO—), 3.77 (s, 6H, CH3) |
| 3 | 421 (M−), 423 (MH+) | δ 7.90 (d, J = 15.9 Hz, 2H), 7.43 (d, J = 8.4 Hz, 2H ), 6.54 (d, |
| | | J = 15.9 Hz, 2H), 6.40-6.19 (m, 4H), 5.76 (s, 1H) 3.90 (s, 6H, |
| | | OCH3), 3.05 (s, 12H, N(CH3)2) |
| 4 | 335 (M−) | δ (in DMSO) 7.77 (m, 6H), 7.14 (m, 2H), 6.84 (m, 4H), 2.75 |
| | | (m, 2H), 1.08 (m, 3H) |
| 5 | 311 (MH−) | δ 7.0 (d, J = 8.4 Hz, 4H), 6.66 (d, J = 8.4 Hz, 4H), 5.50 (s, 1H), |
| | | 2.77 (m, 4H), 2.52 (m, 4H) |
| 6 | 347 (M−) | δ (in CDCl₃/CD₃OD, 9:1, v/v) 7.46 (m, 3H), 7.32 (d, J = 9.3 Hz, |
| | | 2H), 7.17 (d, J = 9.3 Hz, 2H), 6.82 (d, J = 15.9, 2H), 6.67 (m, |
| | | 4H), 2.55 (m, 6H), 1.67 (m, 3H) |
| 7 | 461 (MH−) | δ 8.01 (d, J = 15 Hz, 2H), 7.88 (s, 1H), 7.44 (m, 2 H), 7.01 (d, |
| | | J = 15 Hz, 2H), 6.20 (m, 2H), 3.88 (d, J = 6 Hz, 6 H), 3.03 (d, |
| | | J = 6 Hz, 12H), 2.94 (m, 4H), 1.76 (m, 2H) |
| 8 | 327 (MH−) | δ 6.53 (m, 2H), 4.96 (d, J = 9.9 Hz, 1H), 3.79 (t, 1H), 2.5-2.28 |
| | | (m, 6H), 1.71-1.04 (m, 22H) |
| 9 | 413 (MNa+) | δ 7.63 (d, J = 15.3 Hz, 2H), 7.45 (m, 4H), 6.70-6.65 (m, J = 15.3 |
| | | Hz, 6H), 3.90 (t, J = 7.8 Hz, 1H), 3.02 (m, 12H), 2.03 (m, |
| | | 2H), 0.96 (t, J = 6.0 Hz, 3H) |
| 10 | 403 (MH+), 425 (MNa+) | δ 7.72 (d, J = 15.0 Hz, 1H), 7.65 (s, 1H), 7.50-7.38 (m, 4H), |
| | | 6.92 (d, J = 15.0 Hz, 1H), 6.70 ( m, 4H), 3.03 (d, J = 6.0 Hz, |
| | | 12H), 2.67 (m, 2H), 2.65( m, 2H), 1.79 (m, 2H) |
| 11 | 411 (M−) | δ 8.05 (d, J = 16.5 Hz, 1H), 7.81 (s, 1H), 7.17 (d, J = 16.5 Hz, |
| | | 1H), 7.05-6.96 (m, 3H), 6.88-6.81 (m,3H), 3.86 (d, J = 11.1 Hz, |
| | | 6H), 2.67 (m, 2H), 2.60 (m, 2H), 1.80 (m, 2H) |
| 12 | 423 (MNa+), 399 (MH+) | δ 7.96-7.88 (m, J = 16.2 Hz, 2H), 7.26-6.98 (m, 6H), 6.80 (d, |
| | | J = 16.2 Hz, 2H), 3.81 (d, J = 9.6 Hz, 6H), 3.31 (m, 1H), 1.95 |
| | | (m, 2H), 0.93 (m, 3H) |
| 13 | 449 (MH−) | δ 8.00 (d, J = 15.6 Hz, 2H), 7.44 (d, J = 6.9 Hz, 2H), 6.80 (d, |
| | | J = 15.6 Hz, 2H), 6.25 (m, 4H), 4.03 (m, 1H), 3.88 (m, 6H), |
| | | 3.05 (m, 12H), 2.01 (m, 2H), 0.95 (m, 3H) |
| 14 | 411 (MH−) | δ 8.02 (m, 2H), 7.83 (m, 2H), 7.70 (m, 5H), 6.26 (m, 1H), |
| | | 6.73 (d, J = 16.2 Hz, 2H), 5.90 (s, 1H) |
| 15 | 309 (MH+), 307 (MH−) | δ 7.74 (m, 2H), 7.57 (m, 2H), 7.25 (m, 4H), 7.05 (m, 2H), |
| | | 6.84 (m, 4H) |
| 16 | 335 (MH−) | δ (in CD₃OD) 7.62 (d, J = 15.9 Hz, 2H, Ph—CH—), 7.53 (m, 4H, |
| | | Ph), 6.93 (m, 4H, Ph), 6.51 (d, J = 15.9 Hz, 2H, —CH—), 5.80 (s, |
| | | 1H, —CO—CH—CO—) |
| 17 | n.d. ^{b} | δ 7.70 (d, J = 18.0 Hz, 2H, Ph—CH—), 7.61 (m, 4H, Ph), 7.17 (m, |
| | | 4H, Ph), 6.60 (d, J = 18.0 Hz, 2H, —CH—CO—), 5.87 (s, 1H, |
| | | —CH—), 2.35 (s, 6H, 2x CH3) |
| 18 | 477 (MH+), 475 (MH−) | δ 7.70 (d, J = 18.0 Hz, 2H, Ph—CH—), 7.6 (m, 4H, Ph), 7.17 (m, |
| | | 4H, Ph), 6.60 (d, J= 18.0 Hz, 2H, —CH—CO—), 5.87 (s, 1H, |
| | | —CH—), 1.22 (s, 18H, 2x (CH3)3) |
| 19 | 309 (MH+), 331 (MNa+), 307 | δ 7.50 (d, J = 15.9 Hz, 2H Ph—CH), 7.16 (m, 2H, Ph), 7.00-6.95 |
| | (MH−) | (m, 4H, Ph), 6.78 (m, 2H, Ph), 6.53 (d, J= 15.9 Hz, 2H, |
| | | —CH —CO —), 5.80 (s, 1H, —CH—) |
| 20 | 174 as major peak | δ 7.60 (d, J = 15.6 Hz, 2H, Ph—CH—), 7.45 (m, 4H, Ph), 6.68 (m, |
| | | 4H, Ph), 6.42 (d, J = 15.6 Hz, 2H, —CH—CO—), 5.73 (s, 1H, |
| | | —CH—), 3.03 (s, 12H, 4x CH3) |
| 21 | 369 (MH+), 367 (MH−) | δ 7.40 (d, J = 17.1 Hz; 2H, Ph—CH—), 6.99 (m, 2H, Ph), 6.91 |
| | | (m, 2H, Ph), 6.73 (m, 2H, Ph), 6.34 (d, J = 17.1 Hz, 2H, —CH—), |
| | | 5.70 (s, 1H, —CO—CH—CO—), 3.78 (s, 6H, 2 x CH3) |
| 22 | 367 (MH−) | δ 7.87 (d, J = 16.2 Hz; 2H, Ph—CH—), 7.40 (m, 2H, Ph), 6.77 |
| | | (m, 2H, Ph), 6.63 (d, J = 17.1 Hz, 2H, —CH—), 6.46 (m, 2H, Ph), |
| | | 5.80 (s, 1H, —CO—CH—CO—), 3.85 (s, 6H, 2 x CH3) |
| 23 | 368 (M+) | δ 8.08 (s, 2H), 7.05 (m, 4H), 6.38-6.30 (m, 4H), 3.80 (s, 6H, |
| | | 2x CH3) |
| 24 | 376 (100%), 378 (66%), 377 | δ 7.54 (d, J = 15.9 Hz; 2H, Ph—CH—), 7.40-7.36 (m, 4H, Ph), |
| | (21%) (M⁺), 375 (100%), | 6.33 (s, 2H, Ph), 6.47 (d, J = 15.9 Hz, 2H, —CH—), 5.77 (s, 1H, |
| | 377 (66%), 376 (21%) (MH⁻) | —CO—CH—CO—) |
| 25 | 337 (MH+) | δ 8.05 (d, J = 15.0 Hz, 2H, Ph—CH—), 7.85-7.60 (m, 4H, Ph), |
| | | 7.11-6.70 (m, 4H, Ph), 6.66 (d, J = 15.0 Hz, 2H, —CH—) 6.0 (s, |
| | | 1H), 3.90 (s, 6H, 2x CH3) |
| 26 | 371 (MH−) | δ 7.93 (d, J = 15.0 Hz; 2H, Ph—CH—), 7.40 (m, 2H, Ph), 7.10- |
| | | 6.98 (m, 3H, Ph), 6.87-6.84 (m, 3H, Ph),6.66 (d, J = 15.0 Hz, |
| | | 2H, —CH—), 5.87 (s, 1H, —CO—CH—CO—), 3.88 (s, |
| | | 6H, 2 x CH3) |
| 27 | 275 (MH−) | δ 5.86 (s, 1H), 6.64 (d, J = 15.9 Hz, 2H), 7.4 (m, 6H), 7.57 (m, |
| | | 4H), 7.67 (d, J = 15.9 Hz, 2H) |
| 28 | 303 (MH−) | δ 7.64 (d, J = 15.7 Hz, 2H), 7.27 (s, 1H), 7.21 (d, J = 7.8 Hz, |
| | | 4H), 6.60 (d, J = 16.4 Hz, 2H), 5.83 (s, 1H), 4.65 (d, J = 7.8 4H), |
| | | 2.39 (s, 6H) |
| 29 | n.d. | δ 7.67 (d, J = 8.5 Hz, 4H), 7.60 (d, J = 15.9 Hz, 2H), 7.30 (d, |
| | | J = 8.5 Hz, 4H), 6.90 (d, J = 15.9 Hz, 2H), 6.14 (s, 1H), 2.5 (s, 6H) |
| 30 | 387 (MH−) | δ 7.65 (d, J = 15.9 Hz, 2H), 7.46 (q, J = 17.6 Hz, 5.2 Hz, 8H), |
| | | 6.60 (d, J = 16.15 Hz, 2H), 5.85 (s, 18H) |
| 31 | 370 (MH+) | δ 7.95 (d, J = 18 Hz, 1H), 7.58-7.44 (m, J = 15 Hz, 3H ), 7.05 |
| | | (m, 2H), 6.88 (m, 2H), 6.66 (m, 2H), 6.28 (d, J = 15 Hz, 1H), |
| | | 5.87 (s, 1H), 5.60 (s, 1H),3.88 (s, 3H), 3.03 (s, 6H) |
| 32 | 335 (MH+) | δ (in CD₃OD) 7.57 (d, J = 16.5 Hz, 1H), 7.46-7.41 (m, 4H), |
| | | 7.22 (m, 1H), 6.87-6.80 (m, 4H), 6.18 (d, J = 16.5 Hz, 1H), |
| | | 2.99 (m, 2H), 2.88 (m, 2H) |
| 33 | 507 (MH+) | δ (in CD3OD) 7.76 (d, J = 8.4 Hz, 4H), 7.69 (d, J = 15.9 Hz, |
| | | 2H), 7.26 (d, J = 8.4 Hz, 4H), 6.85 (d, J = 15.9 Hz, 2H), 6.10 |
| | | (s, 1H), 4.25 (d, J = 4.8 Hz, 2H), 2.5 (m, 2H), 1.22-1.19 (m, 12H) |
| 34 | 565 (MH+) | δ (in CD3OD) 7.80(m, 2H), 7.54 (m, 4H), 7.32 (m, 2H), 6.84 |
| | | (m, 4H), 4.41( m, 4H), 3.01 (m, 4H), 2.18 (m, 6H), 1.81 (m, 4H) |
| 35 | 567 (MH+) | δ (in CD3OD) 7.70 (d, J = 16.2 Hz, 2H), 7.43 (s, 2H), 7.30 (m, |
| | | 2H), 7.18 (d, J = .1 Hz, 2H), 6.88 (d, J = 16.2 Hz, 2H), 6.10 |
| | | (s, 1H), 4.27 (m, 2H), 3.91 (s, 6H), 2.50 (m, 2H), 1.24 (m, 12H) |
| 36 | 647 (MNa+) | δ (in CD3OD) 7.68 (d, J = 16.2 Hz, 2H), 7.44 (m, 2H), 7.31 (m, |
| | | 2H), 7.20 (d, J = 8.1 Hz, 2H), 6.89 (d, J = 16.2 Hz, 2H), 6.13 (s, |
| | | 1H), 4.41 (t, J = 6.3 Hz, 2H), 3.96 (t, J = 6.3 Hz, 2H), 3.92 (s, |
| | | 6H), 2.98 (m, 6H), 2.15-1.76 (m, 6H) |
| 37 | 547 (MH+) | δ (in CD30D) 7.81 (d, J = 8.4 Hz, 2H), 7.73 (d, J = 16.5 Hz, |
| | | 2H), 7.55 (d, J = 8.4 Hz, 2H), 7.33 (m, 5H), 4.26 (m, 2H), 2.76 |
| | | (m, 4H), 2.50 (m, 2H), 1.82 (m, 2H), 1.21 (m, 12H) |
| 38 | 605 (MH+) | δ 7.79 (d, J = 8.4 Hz, 2H), 7.70 (d, J= 16.5 Hz, 2H), 7.54 (d, |
| | | J = 8.4 Hz, 2H), 7.32 (m, 5H), 4.96 (m, 2H), 4.39 (m, 4H), 2.99 |
| | | (m, 8H), 2.73 (m, 4H), 2.22-1.53 (m, 6H) |
| 39 | 435 (MH+), 433 (M−) | δ 7.70 (s, 1H), 7.65 (d, J = 8.7 Hz, 1H), 7.21-6.96 (m, 7H), |
| | | 2.77 (m, 2H), 2.68 (m, 14H), 1.77 (m, 2H). |
| 40 | 633 (MH⁺) | (in CD₃OD) δ 7.90-7.42 (m, 9H), 4.58 (m, 2H), 4.57 (m, 2H), |
| | | 3.12 (m, 12H), 2.74-2.58 (m, 4H), 1.28 (m, 2H), 1.24 (m, 12H). |
| 41 | 376 (MH⁺) | (CDCl₃/CD₃OD 30:1) δ 7.65 (s, 1H), 7.67 (m, 1H), 7.42 (m, |
| | | 2H), 6.90 (m, 1H), 6.84-6.77 (m, 6H), 3.14 (s, 6H), 2.69 (m, |
| | | 2H), 2.59 (m, 2H), 1.73 (m, 2H). |
| 42 | 475 (MH⁺), 376 (MH⁺-Val) | (CD₃OD) δ 7.84 (m, 2H), 7.71 (m, 3H), 7.56-7.47 (m, 3H), |
| | | 7.32-7.24 (m, 3H), 4.25 (m, 1H), 2.75 (m, 3H), 2.50 (m, 2H), |
| | | 1.81 (m, 2H), 1.22 (m, 6H). |
| 43 | 407 (M⁻) | δ 7.73 (s, 1H), 7.67 (d, J = 9 Hz, 1H), 7.16 (m, 1H), 7.06-6.93 |
| | | (m, 6H), 3.96 (s, 3H), 3.92 (s, 3H), 2.77 (m, 2H), 2.67 (m, |
| | | 2H), 1.81 (m, 2H). |
| 44 | 607 (MH⁺), 629 (MNa⁺) | (CD₃OD) δ 7.72 (m, 2H), 7.45 (m, 2H), 7.34-7.11 (m, 6H), |
| | | 4.27 (m, 2H), 3.91 (s, 3H), 3.87 (s, 3H), 2.77 (m, 4H), 2.50 |
| | | (m, 3H), 1.81(m, 2H), 1.23 (m, 12H). |
| 45 | 664 (MH⁺) | (CD₃OD) δ 7.72 (m, 2H), 7.46 (s, 1H), 7.34-7.11 (m, 6H), 4.41 |
| | | (t, J = 6 Hz, 4H), 4.05 (t, J = 6 Hz, 4H), 3.93 (s, 3H), 3.88 (s, |
| | | 3H), 3.01 (m, 8H), 2.78 (m, 4H), 2.01 (m, 2H). |
| 46 | 455 (MH ⁺) | δ 7.72 (d, J = 15Hz, 1H), 7.65 (s, 1H), 7.49 (d, J = 9Hz, 2H), |
| | | 7.40 (d, J = 9Hz, 2H), 6.89 (d, J = 15Hz, 1H), 6.56 (m, 4H), 3.36 |
| | | (m, 8H), 2.80 (m, 2H), 2.65 (app t, 2H), 2.04 (m, 8H), 1.80 |
| | | (m, 2H). |
| 47 | 447 (M ⁻) | δ 8.03 (d, J = 9Hz, 1H), 7.98 (m, 2H), 7.90 (d, J = 9H, 2H), |
| | | 7.81 (s, 1H), 7.76 (m, 4H), 7.68 (d, J = 9Hz, 2H), 7.52 (d, J = 9 Hz, |
| | | 2H), 7.13 (d, J = 15Hz, 1H), 6.50 (m, 2H), 2.82 (m, 2H), |
| | | 2.72 (m, 2H), 1.84 (m, 2H). |
| 48 | 487 (MH⁺), 485 (M⁻) | δ 8.03 (d, J = 9Hz, 1H), 7.98 (m, 2H), 7.90 (d, J = 9H, 2H), |
| | | 7.81 (s, 1H), 7.76 (m, 4H), 7.68 (d, J = 9Hz, 2H), 7.52 (d, J = 9 Hz, |
| | | 2H), 7.13 (d, J = 15Hz, 1H), 6.50 (m, 2H), 2.82 (m, 2H), |
| | | 2.72 (m, 2H), 1.84 (m, 2H). |

^{a} Solvent for NMR is CDCl₃ unless indicated otherwise.
^{b} n.d., not determined.

### Example 9

### Evaluate Physico-Chemical and Pharmaceutical Properties of Curcumin Analogs

Chemical and metabolic stability limit the bioavailability and hence usefulness of curcuminoids in vivo. To get around these liabilities, curcuminoids were designed to withstand chemical and metabolic degradation and have longer half-lives. Table 6 shows that compared with curcumin and BDC (1), synthetic analogs possess significantly greater chemical (greater than 60, 90, 120, 180, 240, 360, or 480 minutes half-life in water) and metabolic (>20, 30, 60, 90 or 120 minutes in the HLM assay or >5, 10, 20, 30, 60, 90 or 120, 180, 240 or 300 minutes in the S9) stability. While not expressly tested, it is anticipated that the bioavailability of these analogs will be significantly greater than their parent compounds. Other properties also detract from the potential to use curcumin as a drug to treat AD. Solubility and permeability of curcumin is unfavorable in that the lack of solubility does not allow efficient administration and also affords low bioavailability. The lipophilicity of curcumin also causes extensive protein binding that also limits bioavailability. To circumvent these problems, classes of water-soluble curcumins and BDC analogs were synthesized (Example 8) and tested in vitro (Example 9). By water soluble is meant having a solubility greater than or equal to 0.1 ug/mL) In many cases, the water-soluble curcumin and BDC analogs possessed superior solubility and permeability properties compared with their parent compounds. Finally, anti-oxidant activity is associated with a number of curcumins. In addition to anti-oxidant activity, many of these agents also exhibit pro-oxidant activity and this can be detrimental to the cell. Many of the BDC and curcumin analogs synthesized were designed to remove or decrease anti-oxidant activity and hence provide an agent without the detrimental aspects of the pro-oxidant action (Table 6).

TABLE 6
Physical and Chemical Properties of Selected Curcuminoids.

| | | | | | Anti- | Avg. | |
|---|---|---|---|---|---|---|---|
| | | | | | oxidant | perme- | Avg. |
| | Half life, | | Chemical stability | | activity | ability | Solu- |
| | t_{1/2} (min)^{b} | | (t_{1/2}, min) | | IC₅₀ | (10⁻⁶ | bility |
| Cpd# | HLM | S9 | H₂O | pH 7.4 | (μM) | cm/s) | (μg/mL) |
| 1 | 16.4 | 4.0 | 60.0 | 14.0 | >250 | 305 ± 9 | insol |
| 2 | 40.8 | 10.1 | 73.5 | 15.9 | 31.7 | 844 ± 24 | 9.5 |
| 3 | 63.6 | 39.5 | 19.9 h | 25.5 h | | | 0.73 |
| 4 | | | | | | | |
| 5 | | | | | >250 | | insol |
| 6 | 60.6 | 35.2 | 244.2 | 436.2 | >250 | | |
| 7 | 126.7 | 183.8 | stable^{a} | stable | | 4.8 | |
| 8 | | | | | | | |
| 9 | nd | nd | | | 55.3 | | |
| 10 | 126.6 | 310.7 | stable | stable | 155 | | |
| 11 | | | | | >250 | | |
| 12 | | | | | | | |
| 13 | | | | | 67.4 | | |
| 14 | | | | | | | |
| 15 | | | | | >250 | | |
| 16 | 9.0 | 10.9 | | | | | |
| 17 | | | | | | | |
| 18 | | | | | | | |
| 19 | 13.3 | 6.6 | 22.8 | 66.2 | | | |
| 20 | 41.5 | 22.7 | stable | stable | >250 | | 0.23 |
| 21 | 5.0 | 6.6 | | | | | |
| 22 | 31.5 | 2.4 | | | | | |
| 23 | | | | | | | |
| 24 | | | | | | | |
| 25 | 72.8 | 10.9 | | | | | |
| 26 | 59.2 | 8.2 | stable | stable | >250 | 129 | 0.44 |
| 27 | | | | | | | |
| 28 | | | | | | | |
| 29 | 97.1 | 75.4 | | | | | |
| 30 | | | | | >250 | | |
| 31 | | | | | | | |
| 32 | | | | | | | |
| 33 | 23.8 | 5.9 | 18.9 h | 1.0 h | >250 | 316 | 18.2 |
| 34 | 19.9 | 4.5 | 10.2 | unstable^{b} | >250 | | |
| 35 | 55.8 | 9.0 | 18.2 h | 2.3 h | >250 | 572 | 22.8 |
| 36 | 19.9 | 4.5 | 10.2 | unstable | >250 | 718 | 30.6 |
| 37 | >1 h | 33.2 | 95.9 | <16 | >250 | | |
| 38 | >1 h | 33.6 | unstable | unstable | >250 | | |

Metabolic stability tested in human liver microsomes (HLM) and human S9 fraction.
^{a}stability half-lives greater than 5 hours.
^{b}stability half-lives less than 5 minutes.

Summary, pharmacological development. Natural curcumin BDC 1 is a strong anti-AD and anti-ALS drug lead, but has limitations with regard to stability and solubility and therefore overall bioavailability. Refinement by a recursive approach to improve the physicochemical properties has led to much more potent and drug-like compounds. For example, it was observed that compounds with phenolic electron donating groups increased gene expression associated with Aβ phagocytosis (i.e., MGAT3, TLRs and VDR) relative to BDC. With regard to chemical stability, we recognized that instability arose from lability of the central portion of the molecule. We observed that introduction of ethyl, cyclohexanone, cyclopentanone and pyranone to the central part of the molecule provided increased chemical and metabolic stability. Specifically, the cyclohexyl-containing BDC analog (10) showed markedly greater stability than BDC. Keeping the cyclohexanone group intact and introducing a 4-dimethyl amino group on the phenyl ring afforded the largest up-regulation of MGAT3, TLRs and VDR observed. It has been reported that amino acid prodrugs can greatly enhance the relative oral bioavailability of drugs because of selective uptake transporters in the gut (Dubey et al., Eur J Med Chem 43, 1837, 2008). Once in the blood stream, the prodrugs rapidly revert to the desired parent compound. his process (described in Scheme 5) is remarkably selective and efficient. We developed prodrug curcuminioid derivatives with amino acid-substituted phenyl rings (e.g. 33 and 36), with significantly improved solubility.

Scheme 5. Valine prodrug is stable in the gut→transported to blood→hydrolyzed in blood to active parent compound.

In vivo levels. By combining all of our drug development information, we have obtained highly potent immunomodulatory agents on the basis of in vitro up-regulation of MGAT3 and other genes plus Aβ clearance in AD macrophages, providing neuroprotection in an ex vivo rodent model of amyloid pathology and against oxidative stress in vitro, and suppression of inflammatory cytokines by ALS macrophages. These compounds possess affinity for the VDR target and maintain excellent physicochemical properties. Biopharmaceutical studies of BDC and BDC analogs were done to show that the prodrug approach works. We administered compound 33, and a corresponding prodrug containing also the stabilizing central cyclohexyl (37), to mice and compared with BDC (1). Table 7 shows that 37 attained 73-fold greater serum concentration than BDC (on a molar basis, i.p. dosing). This confirms that the water soluble prodrug is stable in the gut, is transported into the blood, and the valine is spontaneously or enzymatically cleaved to the parent compound (Scheme 5). This provides more than sufficient concentrations for immune stimulation because only nanomolar amounts are required.

TABLE 7
Serum levels of curcumins after oral or i.p administration to mice ^{a}.

| | Oral administration | | I.P. administration | |
|---|---|---|---|---|
| | (25 mg/kg) | | (100 mg/kg) | |
| | μg/ml of | nmol/ml of | μg/ml of | nmol/ml of |
| Compound | serum | serum | serum | serum |
| BDC (1) | 0.01 ± 0.01 | 0.03 ± 0.02 | 0.42 ± 0.15 | 1.36 ± 0.47 |
| 33 ^{b} | 0.00 | 0.00 | 0.27 ± 0.16 | 0.47 ± 0.15 |
| 37 ^{c} | 1.02 ± 0.29 | 1.65 ± 0.26 | 61.57 ± 1.48 | 99.37 ± 1.34 |

^{a} Values are mean of 3 animals;
^{b} Product quantitated in the serum is BDC;
^{c} Product in serum is 6.

### Example 10

### MGAT3 and TLR Transcription in AD Patient and Control Cells Treated with Curcuminoids

MGAT3 and/or TLR transcription were tested by qPCR in human monocyte cells treated with curcuminoids and compared the results to those treated with vehicle (Table 7). As discussed above, activation or up-regulation of macrophage MGAT3 or TLR's results in many functional outcomes, including the enhancement of amyloidosis and removal of Aβ, increased apoptosis, secretion of inflammatory cytokines, and other anti-AD antimicrobial activities. PBMC's from AD patients generally possess down-regulated MGAT3 and TLRs, whereas control PBMC's possess up-regulated MGAT3 and TLRs (Proc Natl Acad Sci USA 104 (2007) 12849). Thus, the ratio of MGAT3 or TLR transcription upon Aβ stimulation of human monocytes versus vehicle treatment provides an indicator and sensitive method to test the in vitro efficacy of drug candidates. Compounds that cause elevated MGAT3 or TLR transcription are predicted to possess promise as anti-AD (and other neurodegenative) diseases. Repeat assays with BDC and certain analogs showed elevated MGAT3 and TLR. Elevated MGAT3 and TLR suggest that compounds that up-regulate MGAT3 and TLRs hold promise for use in drug development of anti-AD agents. The relative biological activity of the curcumin analogs in AD patient and control cells was ascertained in the in vitro Aβ assay described above (see Example 2). The results are shown in Table 8 below.

TABLE 8
Relative ratios of MGAT3 transcription in AD patient/MGAT3
in control PBMC's for tested curcumin analogs.

| Cpd# | MGAT3 | TLRs |
|---|---|---|
| 1 | ++ | TLR 1 ++ |
| | | TLR 2 + |
| | | TLR 3 ++ |
| | | TLR 4 +++ |
| | | TLR 5 +++ |
| | | TLR 6 + |
| | | TLR 7 ++ |
| | | TLR 8 + |
| | | TLR 9 ++ |
| | | TLR 10 ++ |
| 2 | + | nd^{a} |
| 3 | ++ | TLR 2 ++ |
| | | TLR 3 ++ |
| | | TLR 4 ++ |
| 6 | +++ | nd |
| 7 | +++ | nd |
| 10 | +++++ | nd |
| 11 | + | nd |
| 16 | − | nd |
| 17 | − | nd |
| 18 | − | nd |
| 19 | + | nd |
| 20 | ++++ | nd |
| 21 | + | nd |
| 22 | ++ | nd |
| 24 | − | nd |
| 25 | ++++ | TLR 2 +++ |
| | | TLR 3 ++ |
| | | TLR 4 + |
| 26 | +++++ | TLR 2 + |
| | | TLR 3 ++ |
| | | TLR 4 + |
| 27 | + | nd |
| 28 | ++ | nd |
| 29 | ++++ | nd |
| 30 | + | TLR 2 + |
| | | TLR 3 ++ |
| | | TLR 4 + |
| 31 | ++ | TLR 2 ++ |
| | | TLR 3 ++ |
| | | TLR 4 ++ |
| 33 | + | TLR 2 ++ |
| | | TLR 3 ++ |
| | | TLR 4 + |
| 34 | ++ | TLR 2 +++ |
| | | TLR 3 ++ |
| | | TLR 4 + |
| 35 | ++ | TLR 2 +++ |
| | | TLR 4 + |
| 36 | +++++ | nd |
| 37 | ++ | nd |
| 38 | + | nd |

Dash (−) indicates minimal effect; (+) indicates a ratio of 1.0-1.5; (++) indicates a ratio of 1.5-2.0; (+++ to ++++) indicates a ratio of 2.0 and above.
^{a}nd stands for not done.

### Example 11

### Inhibition of Inflammatory Cytokine Production in ALS PBMCs by Curcuminoids

The discovery of inflammatory activation of the immune system by aggregated WT or mutant SOD-1 in ALS patient monocytes/macrophages opens a new strategy for suppressing neurotoxic inflammatory responses by small molecule drugs (e.g curcuminoids). In the ALS spinal cord, it has been shown that macrophages express COX-2 and iNOS and intrude into dying neurons, mast cells are increased, and blood-brain barrier is disrupted, indicating inflammatory pathology. Mutant SOD-1 is present on mitochondria of spinal neurons accessible to the immune system. Thus chemokines may attract macrophages to neurons as shown by immunohistochemistry. It may be that macrophages become stimulated by misfolded, aggregated WT or mutant SOD-1 proteins (possibly through Toll-like receptor-2), and induce pathological signaling leading to NFkB nuclear translocation and induction of IL-1beta and COX-2. The immune response is then broadened through induction of IL-17 by IL-23, stimulating IL-1beta, IL-6, TNF-alpha, NOS-2, and chemokines.

More recently, it was shown that PBMCs isolated from ALS patients, and to a lesser extent from control subjects, strongly express COX-2 and other cytokines after stimulation with aggregated wild-type or mutant SOD-1. Testing inhibitors of signaling pathways leading to production of inflammatory mediators in these cells showed that induction of COX-2 by SOD-1 was blocked by pretreatment with the synthetic curcuminoid 10. It was also shown that mutant SOD-1 induced polarization of ALS PBMCs to the Th17 type, but the induction was inhibited by 10. Further testing showed that curcuminoids were capable of suppressing to different extents the SOD-1 induction of six cytokines in ALS patients' PBMCs (Table 9). The use of other established signaling inhibitors (plus a novel HBRI inhibitor) showed that these effects are most likely mediated through inhibition of NFkB signaling pathways.

TABLE 9
Ability of compounds to inhibit SOD1-induced
cytokine expression in ALS PBMCs.

| Compound # | IL-1β | IL-6 | IL-10 | IL-17 | TNFa | COX-2 |
|---|---|---|---|---|---|---|
| 10 | ++ | + | ++ | ++ | + | +++ |
| 36 | + | n.d. | n.d. | n.d. | n.d. | n.d. |

n.d., not determined.

Together, these observations strongly support the role of innate immune cells and their interactions with spinal cord neurons in ALS pathology, and suggest that novel anti-inflammatory drugs based on curcuminoid compounds administered as a continuing therapy, possibly in combination with other anti-inflammatory drugs, will have a beneficial effect and lead to sustained improvement in ALS patients.

It is understood that the examples and embodiments described herein are for illustrative purposes only and that various modifications or changes in light thereof will be apparent to persons skilled in the art and are to be included within the spirit and purview of this application and scope of the appended claims. All publications, patents, and patent applications cited herein are hereby incorporated by reference in their entireties for all purposes.

1. A method for treatment of neurodegenerative disease comprising administering to a subject in need of such treatment chemically and metabolically stable and soluble a compound of formula (I-III): 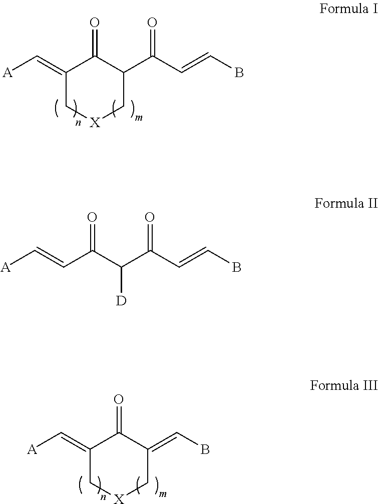
wherein A and B are independently selected from optionally substituted aryl, optionally substituted heterocycle, optionally substituted heteroaryl, or optionally substituted alkyl, wherein A and B are optionally substituted with from 1-5 R groups selected from the group consisting of hydrogen, (C1-C6)alkyl, (C1-C6) alkenyl, (C1-C6) alkynyl, hetero alkyl, halo, (C1-C6) alkoxy, amino, (C1-C6) alkylamino, hydroxy, cyano, nitro, an amino-acid, 5- or 6-member optionally substituted unsaturated, partially unsaturated or saturated heterocyclyl or carbocyclyl optionally substituted with acyl, halo, lower acyl, lower haloakyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;
D is selected from (C1-C6)alkyl, (C1-C6) alkenyl, (C1-C6)alkynyl, heteroalkyl, (C1-C6)alkoxy, amino, (C1-C6)alkylamino, hydroxy, amino acid, optionally substituted unsaturated, partially unsaturated or saturated heterocyclyl or carbocyclyl optionally substituted with acyl, halo, lower acyl, lower haloakyl, oxo, cyano, nitro, carboxyl, amino, amino acid, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;
X is selected from carbon, oxygen, nitrogen, —(CO)N—, —N(CO)—, —(CO)O—, and —O(CO)—, wherein carbon, nitrogen, CON, and NCO are optionally substituted by hydrogen, (C1-C6)alkyl, (C1-C6)alkenyl, (C1-C6)alkynyl, heteroalkyl, (C1-C6)alkoxy, amino, (C1-C6)alkylamino, hydroxyl, amino acid, optionally substituted unsaturated, partially unsaturated or saturated heterocyclyl or carbocyclyl optionally substituted with acyl, halo, lower acyl, lower haloakyl, oxo, cyano, nitro, carboxyl, amino, lower alkoxy, aminocarbonyl, lower alkoxycarbonyl, alkylamino, arylamino, lower carboxyalkyl, lower cyanoalkyl, lower hydroxyalkyl, alkylthio, heteroalkylthio, arylthio, heteroarylthio, heterocyclylthio, carbocyclylthio, lower aralkylthio, alkylsulfinyl, heteroalkylsulfinyl, arylsulfinyl, heteroarylsulfinyl, heterocyclylsulfinyl, carbocyclylsulfinyl, lower aralkylsulfinyl, alkylsulfonyl, heteroalkylsulfonyl, arylsulfonyl, heteroarylsulfonyl, heterocyclylsulfonyl, carbocyclylsulfonyl, lower aralkylsulfonyl, aminosulfinyl, lower N-arylaminosulfinyl, lower arylsulfinyl, and lower N-alkyl-N-arylaminosulfinyl, aminosulfonyl, lower N-arylaminosulfonyl, lower arylsulfonyl, and lower N-alkyl-N-arylaminosulfonyl; and wherein acyl is optionally substituted with a substituent selected from hydrido, alkyl, halo, and alkoxy;
n and m are independently 0, 1, 2, wherein n+m are optionally one or more;
or a tautomer, pharmaceutically acceptable salt, ester and/or prodrug thereof.

2. The method of claim 1, wherein the compound is selected from the group consisting of 4,4′-(3,5-dioxohepta-1,6-diene-1,7-diyl)bis(4,1-phenylene) bis(2-amino-3-methylbutanoate); 2-(4-(Dimethylamino)benzylidene)-3-(4-(dimethylamino)phenyl)-1-hydroxyallylidene)cyclohexanone; 4,4′-(3,5-dioxohepta-1,6-diene-1,7-diyl)bis(2-methoxy-4,1-phenylene) bis(2,6-diaminohexanoate); and 5-(3-(3-(2,6-diaminohexanoyloxy)-4-(dimethylamino)benzylidene)-2-oxocyclohexyl)-3-oxoprop-1-enyl)-2-(dimethylamino)phenyl 2,6-diaminohexanoate.

3-6. (canceled)

7. A method for in vitro screening of a compound for biological or pharmacological activity related to Alzheimer's Disease or ALS comprising the steps of:
(a) incubating cells selected from innate immune cells, monocytes, or macrophages with a compound of formula I, II or III in the presence or absence of amyloid-β (1-42) (Aβ);
and performing at least one of steps (b), (c) or (d):
(b) measuring the expression level of one or more genes associated with stimulation of efficient Aβ phagocytosis;
(c) detecting the amount of amyloid-β (1-42) (Aβ) or other amyloid taken up, neutralized, consumed, and/or phagocytized as an indication of biological or pharmacological activity of the compound; and
(d) detecting the levels of inflammatory cytokines with or without treatment with soluble or aggregated wild-type (WT) or mutant SOD-1.

8. The method of claim 7, wherein step (b) is performed and the expression level of one or more genes selected from MGAT3, a TLR, and VDR is measured.

9. The method of claim 7, wherein step (a) is performed with monocytes, and wherein the monocytes are a human monocytic cell.

10-12. (canceled)

13. A method for predicting efficacy of a drug in an individual, wherein said drug is an MGAT3, VDR and/or TLR modulator and said individual is suffering from or at risk of developing a CNS disorder related to Alzheimer's Disease or ALS amenable to treatment with the drug, said method comprising:
(a) isolating a biological sample from an individual, said biological sample comprising at least one of:
(i) a nucleic acid; and
(ii) a MGAT3 protein, VDR protein or TLR protein; and
(b) analyzing the biological sample to determine the presence or absence of an allele of the MGAT3 gene in the individual, wherein the presence of a specific allele correlates with a specific clinical outcome for treatment of the disorder with the drug;
wherein the drug is a compound of formula I, II, or III.

14-16. (canceled)

17. The method of claim 13, wherein the analyzing step comprises analyzing the nucleic acid from the biological sample to determine at least one specific allele selected from MGAT3, VDR and/or TLR.

18. The method of claim 17, wherein the analyzing step comprises hybridization of the nucleic acid from the biological sample with a second nucleic acid selected from the group consisting of:
(a) a nucleic acid comprising at least 10 to 100 contiguous nucleotides of the nucleotide sequences set forth in SEQ ID Nos.:1-12 comprising at least:
(i) one of the nucleotides at a polymorphic position; and
(ii) a base adjacent thereto; and
(b) a nucleic acid that is fully complementary to the nucleic acid of (a).

19. The method of claim 18, wherein said second nucleic acid is conjugated to a detectable marker.

20. The method of claim 13, further comprising determining the MGAT3, VDR and/or TLR genotype.

21. A method for predicting efficacy of a candidate agent for the treatment of a CNS disorder related to Alzheimer's Disease or ALS, wherein said candidate agent is a derivative of a predetermined therapeutic agent for the treatment of the disorder, said method comprising:
(a) contacting a sample of the MGAT3, VDR or TLR protein from an AD or ALS individual with the candidate agent;
(b) contacting a sample of the MGAT3, VDR or TLR protein from a healthy individual with the predetermined therapeutic agent;
wherein said contacting in (a) or (b) occurs under conditions suitable for MGAT3, VDR and/or TLR functional activity;
(c) determining for each of the samples the level of MGAT3 and/or TLR functional activity; and
(d) comparing the level of MGAT3, VDR and/or TLR functional activity in the sample from the AD or ALS individual with the level of MGAT3, VDR and/or TLR functional activity in the sample from the healthy individual;
wherein a greater level of MGAT3, VDR and/or TLR functional activity in the sample from the AD or ALS individual relative to the MGAT3, VDR and/or TLR functional activity in the sample from the healthy individual is indicative of the efficacy of the candidate agent.

22. The method of claim 21, wherein the MGAT3, VDR or TLR protein is a variant of MGAT3, VDR, TLR.

23. The method of claim 21, wherein the predetermined therapeutic agent has a structure selected from formula I, II or III.

24. The method of claim 23, wherein the predetermined therapeutic agent has a structure of formula II.

25. The method of claim 21, wherein the candidate agent has been modified to incorporate an MGAT3, VDR and/or TLR inducer moiety.

26. (canceled)

27. The method of claim 21, wherein determining the level of MGAT3, VDR and/or TLR functional activity comprises detecting the level of an N-glycated peptide or protein as a function of the drug candidate in a sample.

28. (canceled)

29. The method of claim 1, wherein the neurodegenerative disease is selected from Alzheimer's disease (AD) and Amyotrophic lateral sclerosis (ALS).

30. (canceled)

31. The method of claim 1, wherein A and B are aryl or heteroaryl substituted by R₁, R₂, R₃, R₄, and R₅, wherein R₁, R₂, R₃, R₄, R₅ are independently selected from hydrogen, Cl, Br, I, —OR₆, an amino acid attached through its amino or acid function, —OC(O)R₆, OC(O)NR₇R₈, —C(O)R₉, —CN, —NR₁₀R₁₁, —SR₁₂, —S(O)R₁₁, —S(O)₂R₁₄, —C(O)OR₁₅, —S(O)₂NR₁₆R₁₇, and —R₁₈NR₁₉R₂₀ wherein R₆, R₇, R₈, R₉, R₁₀, R₁₁, R₁₂, R₁₃, R₁₄, R₁₅, R₁₆, R₁₇, R₁₈, R₁₉, and R₂₀ are the same or different and are selected from hydrogen or branched or unbranched alkyl groups comprising one to eight carbon atoms optionally linked to each other to form an additional ring.

32-44. (canceled)

45. The method of claim 21, wherein the compound is selected from the group consisting of:
6-hydroxyhepta-2,4-dienoyl)-6-(4-hydroxypent-2-enylidene)cyclohexanone;
(3-methylbutylidene)-5-methylhex-2-enoyl)cyclohexanone;
3-(4-hydroxycyclohexyl)acryloyl)-6-((4-hydroxycyclohexyl)methylene)cyclohexanone;
1-(4-hydroxybenzyl)-N-isopentylpiperidine-3-carboxamide;
N-(3-hydroxy-3-methylbutyl)-1-(4-hydroxybenzyl)piperidine-3-carboxamide;
4,4′-3-hydroxy-5-oxohepta-1,3,6-triene-1,7-diyl)bis(4,1-phenylene)bis(1-aminocyclo propanecarboxylate);
4-(3-(4-(2-amino-3-methylbutanoyloxy)-3-methoxybenzylidene)-2-oxocyclohexylidene)-3-hydroxyprop-1-enyl)-2-methoxyphenyl 2-amino-3-methylbutanoate;
4-(3-(4-(2,6-diaminohexanoyloxy)-3-methoxybenzylidene)-2-oxocyclohexylidene)-3-hydroxyprop-1-enyl)-2-methoxyphenyl 2,6-diaminohexanoate;
2-(4-(dimethylamino)benzylidene)-6-(3-(4-hydroxyphenyl)acryloyl)cyclohexanone;
2-(4-(dimethylamino)-3-hydroxybenzylidene)-6-(3-(4-(dimethylamino)-3-hydroxyphenyl)acryloyl)cyclohexanone;
4-(3-(3-(4-(dimethylamino)benzylidene)-2-oxocyclohexyl)-3-oxoprop-1-enyl)phenyl-2-amino-3-methylbutanoate;
4-(3-(3-(4-(dimethylamino)benzylidene)-2-oxocyclohexyl)-3-oxoprop-1-enyl)phenyl-2,6-diaminohexanoate;
5-(3-(3-(3-(2-amino-3-methylbutanoyloxy)-4-(dimethylamino)benzylidene)-2-oxocyclohexyl)-3-oxoprop-1-enyl)-2-(dimethylamino)phenyl 2-amino-3-methylbutanoate;
5-(3-(3-(3-(2,6-diaminohexanoyloxy)-4-(dimethylamino)benzylidene)-2-oxocyclohexyl)-3-oxoprop-1-enyl)-2-(dimethylamino)phenyl 2,6-diaminohexanoate.

45-47. (canceled)


SEQUENCE LISTING (32 sequences)

SEQ ID NO: 1-12 - skipped (12 entries)

SEQ ID NO: 13 - DNA, 17 bp, Homo sapiens
cggacgccagcatctcc

SEQ ID NO: 14 - DNA, 23 bp, Homo sapiens
agggctgagggaggccagttctc

SEQ ID NO: 15 - DNA, 20 bp, Mus musculus
cggacgatgggatgaagatg

SEQ ID NO: 16 - DNA, 20 bp, Mus musculus
aggccagttctctcgggaag

SEQ ID NO: 17 - DNA, 20 bp, Homo sapiens
gcgttctctcaggtgactgc

SEQ ID NO: 18 - DNA, 21 bp, Homo sapiens
cccacaggtaccttcacttgg

SEQ ID NO: 19 - DNA, 21 bp, Mus musculus
ttcagtcttcctaggctggtg

SEQ ID NO: 20 - DNA, 20 bp, Mus musculus
acacatctcctgccagtgac

SEQ ID NO: 21 - DNA, 23 bp, Homo sapiens
aaaggaaaggctagcagtcatcc

SEQ ID NO: 22 - DNA, 20 bp, Homo sapiens
cagtgcacttggtggtggag

SEQ ID NO: 23 - DNA, 21 bp, Mus musculus
tcatttcgttatcacacacca

SEQ ID NO: 24 - DNA, 22 bp, Mus musculus
aattgaatccagattttgctca

SEQ ID NO: 25 - DNA, 20 bp, Homo sapiens
atcccctgaggcatttaggc

SEQ ID NO: 26 - DNA, 21 bp, Homo sapiens
tgccccatcttcaattgtctg

SEQ ID NO: 27 - DNA, 20 bp, Mus musculus
gctcctggctaggactctga

SEQ ID NO: 28 - DNA, 20 bp, Mus musculus
tgtcatcagggactttgctg

SEQ ID NO: 29 - DNA, 21 bp, Homo sapiens
cagcatccaaaaggtcattgg

SEQ ID NO: 30 - DNA, 22 bp, Homo sapiens
caatggcacttgacttcagcag

SEQ ID NO: 31 - DNA, 20 bp, Mus musculus
ccaaaaggtcatcggctttg

SEQ ID NO: 32 - DNA, 19 bp, Mus musculus
ggagcgcaacatgatcacc